(19) United States
(12) Patent Application Publication
(10) Pub. No.: US 2005/0019901 A1
(43) Pub. Date: Jan. 27, 2005

(54) Methods for synthesis of bio-active nanoparticles and nanocapsules for use in optical bio-disc assays and disc assembly including same

(76) Inventors: Evgenia Matveeva, Irvine, CA (US); Ramoncito Magpantay Valencia, Aliso Viejo, CA (US); Martina Elisabeth Werner, Aliso Viejo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/356,666

(22) Filed: Jan. 30, 2003

Related U.S. Application Data
(63) Continuation-in-part of application No. 10/150,702, filed on May 17, 2002.
(60) Provisional application No. 60/353,949, filed on Jan. 31, 2002.

(51) Int. Cl. G07D 1/06; C12M 1/34; B29C 35/08

(52) U.S. Cl. 435/287.2; 264/425

(57) ABSTRACT

Optical bio-disc assays and synthesis of bio-active nanoparticles and nanocapsules for use therewith. Related methods for synthesis of polymeric nanoparticles for use in disc assays include forming reverse micelles having an outer non-polar shell and an inner polar cavity and solubilizing in the reverse micelles a polymerizing mixture including monomers, co-monomers, weakly polar monomers, and/or polymerizable surfactants. This may also include an initiator of polymerization. The methods also include polymerizing the mixture. The invention is also directed to the use of the nanoparticles and nanocapsules in optical bio-disc assays for the detection of analytes including nucleic acid sequences. Related optical assay discs and disc systems are also provided.

### CROSS-REFERENCE TO RELATED APPLICATIONS

The present application is a continuation in part of U.S. patent application Ser. No. 10/150,702 filed May 16, 2002.

This application also claims the benefit of priority from U.S. Provisional Application Ser. No. 60/353,949 filed on Jan. 31, 2002 which is herein incorporated by reference in its entirety.

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND OF THE INVENTION

1. Field of the Invention

This invention relates in general to chemical and biological assays and, in particular, to synthesis of nanoparticles and nanocapsules for use in optical bio-disc assays. More specifically, but without restriction to the particular embodiments hereinafter described in accordance with the best mode of practice, this invention relates to the synthesis of bio-active nanoparticles and nanocapsules for use in disc assays and optical analysis discs adapted for use therewith.

2. Discussion of the Background Art

It is known in the prior art that microparticles can be synthesized by polymerization from monomers to give linear or cross-linked polymer particles. Polymerization is initiated by an initiator and occurs fast as a chain reaction. As a result, particles of very big sizes and even continuous gel-like structures can form if the reaction is not stopped after some time. Even when the polymerization reaction is stopped, the resulting particles significantly differ in size and shape. It is an object of the present invention to provide methods for synthesis of polymer nanoparticles or nanocapsules of uniform size and shape and to provide suitable means for disc assays and disc analysis for analyte detection.

### SUMMARY OF THE INVENTION

The present invention is directed to the synthesis of nanoparticles or nanocapsules for use in disc assays and optical analysis discs adapted for use therewith.

More specifically, the present invention is directed to a method for preparing synthetic nanoparticles comprising the steps of forming reverse micelles (RM) having an outer non-polar shell and an inner polar cavity by contacting one or more surfactants or emulsifiers with a non-polar organic solvent. Once the micelles are formed a polymerizing mixture comprising one or more monomers or co-monomers and an initiator of polymerization is solubilized into the micelle. The mixture is then polymerized. In another embodiment of the present invention, the surfactants or emulsifiers are in part or totally polymeric surfactants that are cross-linked during polymerization.

The present invention is also directed to a method for preparing labeled synthetic nanoparticles including the steps of forming reverse micelles having an outer non-polar shell and an inner polar cavity by contacting one or more surfactants or emulsifiers with non-polar organic solvent. Once the micelles are formed a polymerizing mixture including one or more monomers or co-monomers, an initiator of polymerization and a label including, for example absorbing, luminescent, or fluorescent dyes or particles is incorporated into the micelle. The mixture is then polymerized.

The present invention is further directed to a method for preparing synthetic semiconductor nanoparticles, including the steps of forming reverse micelles having an outer non-polar shell and an inner polar cavity by contacting one or more surfactants or emulsifiers with non-polar organic solvent. The next steps include solubilizing into the reverse micelles a polymerizing mixture having one or more monomers or co-monomers, an initiator of polymerization and metal semi-conductor material. The mixture is then polymerized. Optionally, the surfactants or emulsifiers may be, in part or totally, polymeric surfactants that are cross-linked during polymerization.

The present invention is still further directed to method for preparing synthetic magnetic nanoparticles, including the concomitant or sequential steps of forming reverse micelles having an outer non-polar shell and an inner polar cavity by contacting one or more surfactants or emulsifiers with non-polar organic solvent. In another embodiment of the present invention, the surfactants or emulsifiers may be in part or totally polymeric surfactants that are cross-linked during polymerization. The next step in this method of the present invention, includes solubilizing a polymerizing mixture in the reverse micelles. The polymerizing mixture may include one or more monomers or co-monomers and an initiator of polymerization. The solubilizing step is then followed by adding magnetic particles into the micelles. These magnetic particles are incorporated into the nanoparticles during polymerization. The mixture, including the nanoparticles that have been incorporated in to the micelles, is then polymerized. The method for preparing synthetic magnetic nanoparticles may further include the step of coating the synthetic magnetic nanoparticles with a bio-compatible polymer.

In all the methods according to the present invention, the polymerizing mixture may be comprised of acrylic or methacrylic compounds forming a linear or cross-linked polymer and optionally additional cross-linking agents. The surfactants or emulsifiers are selected from anionic, cationic, and non-ionic surfactants. The non-polar organic solvent is a hydrocarbon.

Further embodiments of the present invention include synthetic nanoparticles adapted for use in disc assays having size of about 1 to 1000 nanometers obtained according to the above-recited methods. The synthetic nanoparticles may be synthesized from acrylic or methacrylic linear or cross-linked polymers, optionally further including cross-linking agents.

In yet another embodiment of the present invention, the synthetic nanoparticles comprise a label absorbing, luminescing or fluorescing molecules having absorbance and/or emission properties at suitable wavelength detectable by an optical disc reader.

In a still different embodiment of the present invention, the nanoparticles are synthetic semiconductor nanoparticles or synthetic magnetic nanoparticles adapted for use in disc assays having size of about 1 to 1000 nanometers and obtained according to the above-recited methods. These magnetic nanoparticles, are optionally coated with bio-compatible polymer.

Still further objects of the present invention are methods for immobilizing a bio-active substance into synthetic nanoparticles including the steps of (1) forming reverse micelles having an outer non-polar shell and an inner polar cavity by mixing surfactants or emulsifiers with a non-polar organic solvent, (2) solubilizing a polymerizing mixture including one or more monomers or co-monomers, an initiator of polymerization and the biologically active substance into the reverse micelles, and (3) polymerizing the mixture. The bio-active substance may also be immobilized into labeled synthetic nanoparticles according to another method including the steps of forming reverse micelles having an outer non-polar shell and an inner polar cavity by contacting one or more surfactants or emulsifiers with a non-polar organic solvent; solubilizing a polymerizing mixture including one or more monomers or co-monomers, an initiator of polymerization, the biologically active substance and one or more labels in the reverse micelles; and polymerizing the mixture. In some embodiments, the labels may be dyes that preferably absorb or fluoresce at a wavelength detectable using an optical disc drive.

The present invention is also directed to methods for immobilizing a bio-active substance into synthetic semiconductor nanoparticles including the steps of forming reverse micelles having an outer non-polar shell and an inner polar cavity by contacting one or more surfactants or emulsifiers with non-polar organic solvent; and solubilizing a polymerizing mixture including one or more monomers or co-monomers, an initiator of polymerization, and a biologically active substance into the reverse micelles. The next step in this method is the addition of a metal semi-conductor material into the reverse micelles and polymerizing the resulting mixture.

Further embodiments of the present invention are methods for immobilizing a bio-active substance into synthetic magnetic nanoparticles. These include the steps of forming reverse micelles having an outer non-polar shell and an inner polar cavity by contacting one or more surfactants or emulsifiers with a non-polar organic solvent; incorporating a polymerizing mixture comprising one or more monomers or co-monomers, an initiator of polymerization, and a biologically active substance into the reverse micelles; adding to the reverse micelles magnetic nanoparticles; and polymerizing the resulting mixture.

An alternative method for immobilizing a bio-active substance onto synthetic magnetic nanoparticles includes the steps of coating the synthetic magnetic nanoparticles, free of bio-active substance, with a bio-compatible polymer and conjugating the biologically active substance to the bio-compatible polymer.

In all the above mentioned methods the biologically active substance may include proteins, antigens, antibodies, enzymes, drugs, and functionally active subunits, parts and mixtures thereof. The nanoparticles, nanospheres, or complexes obtained with the above-recited methods, including nanoparticles with a bio-active substances, labeled nanoparticles, semiconductor nanoparticles, magnetic nanoparticles, or magnetic coated nanoparticles, are additional objects of the invention.

Another embodiment of the present invention is a method for making a nanocapsule for use in optical bio-disc assays. This method includes the steps of forming reverse micelles having an outer non-polar shell and an inner polar cavity by mixing micelle-forming surfactants with a non-polar organic solvent; adding weakly polar monomers that solubilized near the shell of the reverse micelles; solubilizing in the reverse micelles an initiator of polymerization; and polymerizing the weakly polar monomers to thereby form the nanocapsule.

The present invention is further directed to optical bio-disc assays for analyte detection including the step of using the above-described nanoparticles, nanospheres, nanocapsules, or complexes in chemical, biological, biochemical, immunochemical, and biomedical assays in optical analysis discs.

The present invention also includes a method of using the above-mentioned nanocapsules or nanoparticles to test for the presence of a target nucleic acid in a test sample. This method include the steps of providing a bio-disc that includes a substantially circular substrate having a center and an outer edge, a target zone disposed between the center and the outer edge, at least one strand of capture DNA attached to the substrate in target zone, the capture DNA and the target nucleic acid having at least some complementary sequence. The method continues with the steps of depositing the test sample on the target zone; allowing any target nucleic acid present in the test sample to hybridize with the capture-DNA; attaching a signal DNA onto the nanocapsule or nanoparticle; depositing the nanocapsule or nanoparticle on the target zone; and hybridizing the signal DNA to the target nucleic acid such that the nanocapsule or nanoparticle is immobilized within the target zone. This method further includes washing the target zone to remove any unbound nanocapsule; depositing onto the target zone at least one enzyme substrate that reacts with the enzyme inside the bio-active nanoparticle to produce at least one detectable signal; and detecting any signal in the target zone to thereby determine whether target-nucleic acid is present in the test sample.

The present is further directed to an optical assay disc implemented to perform any of the above methods, use of such discs in performing any of these methods, and the manufacturing or assemblying of these specific optical disc assemblies as made to perform any of the above methods or assays.

This invention or different aspects thereof may be readily implemented in, adapted to, or employed in combination with the discs, assays, and systems disclosed in the following commonly assigned and co-pending patent applications: U.S. patent application Ser. No. 09/378,878 entitled “Methods and Apparatus for Analyzing Operational and Non-operational Data Acquired from Optical Discs” filed Aug. 23, 1999; U.S. Provisional Patent Application Ser. No. 60/150,288 entitled “Methods and Apparatus for Optical Disc Data Acquisition Using Physical Synchronization Markers” filed Aug. 23, 1999; U.S. patent application Ser. No. 09/421,870 entitled “Trackable Optical Discs with Concurrently Readable Analyte Material” filed Oct. 26, 1999; U.S. patent application Ser. No. 09/643,106 entitled “Methods and Apparatus for Optical Disc Data Acquisition Using Physical Synchronization Markers” filed Aug. 21, 2000; U.S. patent application Ser. No. 09/999,274 entitled “Optical Biodiscs with Reflective Layers” filed Nov. 15, 2001; U.S. patent application Ser. No. 09/988,728 entitled “Methods and Apparatus for Detecting and Quantifying Lymphocytes with Optical Biodiscs” filed Nov. 20, 2001; U.S. patent application Ser. No. 09/988,850 entitled “Methods and Apparatus for Blood Typing with Optical Bio-discs” filed November, 19, 2001; U.S. patent application Ser. No. 09/989,684 entitled “Apparatus and Methods for Separating Agglutinants and Disperse Particles” filed Nov. 20, 2001; U.S. patent application Ser. No. 09/997,741 entitled “Dual Bead Assays Including Optical Biodiscs and Methods Relating Thereto” filed Nov. 27, 2001; U.S. patent application Ser. No. 09/997,895 entitled “Apparatus and Methods for Separating Components of Particulate Suspension” filed Nov. 30, 2001; U.S. patent application Ser. No. 10/005,313 entitled “Optical Discs for Measuring Analytes” filed Dec. 7, 2001; U.S. patent application Ser. No. 10/006,371 entitled “Methods for Detecting Analytes Using Optical Discs and Optical Disc Readers” filed Dec. 10, 2001; U.S. patent application Ser. No. 10/006,620 entitled “Multiple Data Layer Optical Discs for Detecting Analytes” filed Dec. 10, 2001; U.S. patent application Ser. No. 10/006,619 entitled “Optical Disc Assemblies for Performing Assays” filed Dec. 10, 2001; U.S. patent application Ser. No. 10/020,140 entitled “Detection System For Disk-Based Laboratory and Improved Optical Bio-Disc Including Same” filed Dec. 14, 2001; U.S. patent application Ser. No. 10/035,836 entitled “Surface Assembly for Immobilizing DNA Capture Probes and Bead-Based Assay Including Optical Bio-Discs and Methods Relating Thereto” filed Dec. 21, 2001; U.S. patent application Ser. No. 10/038,297 entitled “Dual Bead Assays Including Covalent Linkages for Improved Specificity and Related Optical Analysis Discs” filed Jan. 4, 2002; U.S. patent application Ser. No. 10/043,688 entitled “Optical Disc Analysis System Including Related Methods for Biological and Medical Imaging” filed Jan. 10, 2002; U.S. Provisional Application Ser. No. 60/348,767 entitled “Optical Disc Analysis System Including Related Signal Processing Methods and Software” filed Jan. 14, 2002 U.S. patent application Ser. No. 10/086,941 entitled “Methods for DNA Conjugation Onto Solid Phase Including Related Optical Biodiscs and Disc Drive Systems” filed Feb. 26, 2002; U.S. patent application Ser. No. 10/087,549 entitled “Methods for Decreasing Non-Specific Binding of Beads in Dual Bead Assays Including Related Optical Biodiscs and Disc Drive Systems” filed Feb. 28, 2002; U.S. patent application Ser. No. 10/099,256 entitled “Dual Bead Assays Using Cleavable Spacers and/or Ligation to Improve Specificity and Sensitivity Including Related Methods and Apparatus” filed Mar. 14, 2002; U.S. patent application Ser. No. 10/099,266 entitled “Use of Restriction Enzymes and Other Chemical Methods to Decrease Non-Specific Binding in Dual Bead Assays and Related Bio-Discs, Methods, and System Apparatus for Detecting Medical Targets” also filed Mar. 14, 2002; U.S. patent application Ser. No. 10/121,281 entitled “Multi-Parameter Assays Including Analysis Discs and Methods Relating Thereto” filed Apr. 11, 2002; U.S. patent application Ser. No. 10/150,575 entitled “Variable Sampling Control for Rendering Pixelization of Analysis Results in a Bio-Disc Assembly and Apparatus Relating Thereto” filed May 16, 2002; U.S. patent application Ser. No. 10/150,702 entitled “Surface Assembly For Immobilizing DNA Capture Probes in Genetic Assays Using Enzymatic Reactions to Generate Signals in Optical Bio-Discs and Methods Relating Thereto” filed May 16, 2002; U.S. patent application Ser. No. 10/194,418 entitled “Optical Disc System and Related Detecting and Decoding Methods for Analysis of Microscopic Structures” filed Jul. 12, 2002; U.S. patent application Ser. No. 10/194,396 entitled “Multi-Purpose Optical Analysis Disc for Conducting Assays and Various Reporting Agents for Use Therewith” also filed Jul. 12, 2002; U.S. patent application Ser. No. 10/199,973 entitled “Transmissive Optical Disc Assemblies for Performing Physical Measurements and Methods Relating Thereto” filed Jul. 19, 2002; U.S. patent application Ser. No. 10/201,591 entitled “Optical Analysis Disc and Related Drive Assembly for Performing Interactive Centrifugation” filed Jul. 22, 2002; U.S. patent application Ser. No. 10/205,011 entitled “Method and Apparatus for Bonded Fluidic Circuit for Optical Bio-Disc” filed Jul. 24, 2002; U.S. patent application Ser. No. 10/205,005 entitled “Magnetic Assisted Detection of Magnetic Beads Using Optical Disc Drives” also filed Jul. 24, 2002; U.S. patent application Ser. No. 10/230,959 entitled “Methods for Qualitative and Quantitative Analysis of Cells and Related Optical Bio-Disc Systems” filed Aug. 29, 2002; U.S. patent application Ser. No. 10/233,322 entitled “Capture Layer Assemblies for Cellular Assays Including Related Optical Analysis Discs and Methods” filed Aug. 30, 2002; U.S. patent application Ser. No. 10/236,857 entitled “Nuclear Morphology Based Identification and Quantification of White Blood Cell Types Using Optical Bio-Disc Systems” filed Sep. 6, 2002; U.S. patent application Ser. No. 10/241,512 entitled “Methods for Differential Cell Counts Including Related Apparatus and Software for Performing Same” filed Sep. 11, 2002; U.S. patent application Ser. No. 10/279,677 entitled “Segmented Area Detector for Biodrive and Methods Relating Thereto” filed Oct. 24, 2002; U.S. patent application Ser. No. 10/293,214 entitled “Optical Bio-Discs and Fluidic Circuits for Analysis of Cells and Methods Relating Thereto” filed on Nov. 13, 2002; U.S. patent application Ser. No. 10/298,263 entitled “Methods and Apparatus for Blood Typing with Optical Bio-Discs” filed on Nov. 15, 2002; U.S. patent application Ser. No. 10/307,263 entitled “Magneto-Optical Bio-Discs and Systems Including Related Methods” filed Nov. 27, 2002; U.S. patent application Ser. No. 10/341,326 entitled “Method and Apparatus for Visualizing Data” filed Jan. 13, 2003; U.S. patent application Ser. No. 10/345,122 entitled “Methods and Apparatus for Extracting Data From an Optical Analysis Disc” filed on Jan. 14, 2003; U.S. patent application Ser. No. 10/347,155 entitled “Optical Discs Including Equi-Radial and/or Spiral Analysis Zones and Related Disc Drive Systems and Methods” filed on Jan. 15, 2003; U.S. patent application Ser. No. 10/347,119 entitled “Bio-Safe Dispenser and Optical Analysis Disc Assembly” filed Jan. 17, 2003; U.S. patent application Ser. No. ______ entitled “Multi-Purpose Optical Analysis Disc for Conducting Assays and Related Methods for Attaching Capture Agents” filed on Jan. 21, 2003; U.S. patent application Ser. No. ______ entitled “Processes for Manufacturing Optical Analysis Discs with Molded Microfluidic Structures and Discs Made According Thereto” filed on Jan. 21, 2003; U.S. patent application Ser. No. ______ entitled “Methods for Triggering Through Disc Grooves and Related Optical Analysis Discs and System” filed on Jan. 23, 2003; U.S. patent application Ser. No. ______ entitled “Bio-Safety Features for Optical Analysis Discs and Disc System Including Same” filed on Jan. 23, 2003; U.S. patent application Ser. No. ______ entitled “Manufacturing Processes for Making Optical Analysis Discs Including Successive Patterning Operations and Optical Discs Thereby Manufactured: filed on Jan. 24, 2003; U.S. patent application Ser. No. ______ entitled “Processes for Manufacturing Optical Analysis Discs with Molded Microfluidic Structures and Discs Made According Thereto” filed on Jan. 27, 2003; and U.S. patent application Ser. No. ______ entitled “Method and Apparatus for Logical Triggering” filed on Jan. 28, 2003. All of these applications are herein incorporated by reference in their entireties. They thus provide background and related disclosure as support hereof as if fully repeated herein.

The above described methods and apparatus according to the present invention as disclosed herein can have one or more advantages which include, but are not limited to, simple and quick on-disc processing without the necessity of an experienced technician to run the test, small sample volumes, use of inexpensive materials, and use of known optical disc formats and drive manufacturing. These and other features and advantages will be better understood by reference to the following detailed description when taken in conjunction with the accompanying drawing figures, technical examples, and claims.

### BRIEF DESCRIPTION OF THE DRAWINGS

Further objects of the present invention together with additional features contributing thereto and advantages accruing therefore will be apparent from the following description of the preferred embodiments of the invention which are shown in the accompanying drawing figures with like reference numerals indicating like components throughout, wherein:

FIG. 1 is a pictorial representation of a bio-disc system according to the present invention;

FIG. 2 is an exploded perspective view of a reflective bio-disc as utilized in conjunction with the present invention;

FIG. 3 is a top plan view of the disc shown in FIG. 2;

FIG. 4 is a perspective view of the disc illustrated in FIG. 2 with cut-away sections showing the different layers of the disc;

FIG. 5 is an exploded perspective view of a transmissive bio-disc as employed in conjunction with the present invention;

FIG. 6 is a perspective view representing the disc shown in FIG. 5 with a cut-away section illustrating the functional aspects of a semi-reflective layer of the disc;

FIG. 7 is a graphical representation showing the relationship between thickness and transmission of a thin gold film;

FIG. 8 is a top plan view of the disc shown in FIG. 5;

FIG. 9 is a perspective view of the disc illustrated in FIG. 5 with cut-away sections showing the different layers of the disc including the type of semi-reflective layer shown in FIG. 6;

FIG. 10 is a perspective and block diagram representation illustrating the system of FIG. 1 in more detail;

FIG. 11 is a partial cross sectional view taken perpendicular to a radius of the reflective optical bio-disc illustrated in FIGS. 2, 3, and 4 showing a flow channel formed therein;

FIG. 12 is a partial cross sectional view taken perpendicular to a radius of the transmissive optical bio-disc illustrated in FIGS. 5, 8, and 9 showing a flow channel formed therein and a top detector;

FIG. 13 is a partial longitudinal cross sectional view of the reflective optical bio-disc shown in FIGS. 2, 3, and 4 illustrating a wobble groove formed therein;

FIG. 14 is a partial longitudinal cross sectional view of the transmissive optical bio-disc illustrated in FIGS. 5, 8, and 9 showing a wobble groove formed therein and a top detector;

FIG. 15 is a view similar to FIG. 11 showing the entire thickness of the reflective disc and the initial refractive property thereof;

FIG. 16 is a view similar to FIG. 12 showing the entire thickness of the transmissive disc and the initial refractive property thereof;

FIG. 17 is a pictorial graphical representation of the transformation of a sampled analog signal to a corresponding digital signal that is stored as a one-dimensional array;

FIG. 18 is a perspective view of an optical disc with an enlarged detailed view of the indicated section showing a captured white blood cell positioned relative to the tracks of the bio-disc yielding a signal-containing beam after interacting with an incident beam;

FIG. 19A is a graphical representation of a white blood cell positioned relative to the tracks of an optical bio-disc according to the present invention;

FIG. 19B is a series of signature traces derived from the white blood cell of FIG. 19A according to the present invention;

FIG. 20 is a graphical representation illustrating the relationship between FIGS. 20A, 20B, 20C, and 20D;

FIGS. 20A, 20B, 20C, and 20D, when taken together, form a pictorial graphical representation of transformation of the signature traces from FIG. 19B into digital signals that are stored as one-dimensional arrays and combined into a two-dimensional array for data input;

FIG. 21 is a logic flow chart depicting the principal steps for data evaluation according to processing methods and computational algorithms related to the present invention;

FIG. 22 illustrates the aggregation of surfactants forming normal micelles in an aqueous solution;

FIG. 23 illustrates the aggregation of surfactants forming reverse micelles (RM) in a non-polar organic solvent;

FIG. 24 reports the most common RM-forming surfactants;

FIG. 25 is a graphical representation of the relationship between the size of the reverse micelles and their hydration degree;

FIG. 26 is a schematic illustration of one embodiment of the formation of a nanoparticle in a reverse micelle system;

FIG. 27 is a schematic illustration of the synthesis of a nanocapsule using micelle-forming polymer conjugated surfactants that cross-link with monomer cross-linking agents during polymerization;

FIG. 28A is a schematic representation of a reverse micelle containing an entrapped water-soluble substance;

FIG. 28B is a schematic representation of the formation of a nanoparticle containing an immobilized substance;

FIG. 28C is a schematic representation of the formation of a nanocapsule containing a water-soluble substance;

FIG. 28D is a schematic representation of the formation of a hydrophobized nanocapsule containing a water-soluble substance;

FIG. 28E is a schematic representation of the formation of a surface-modified (hydrophobized) nanoparticle containing an immobilized substance;

FIG. 29A is an illustration of the formation, isolation, and dissolution of the nanoparticle of FIG. 28B into an aqueous solution and in a reverse micelle in an organic solvent;

FIG. 29B is an illustration of the formation, isolation, and dissolution of the hydrophobized nanoparticle of FIG. 28E into a micelle double layer in an aqueous solution and in a reverse micelle in an organic solvent;

FIGS. 30A-30F are pictorial representations of various chemical elements utilized in performing immunoassays;

FIGS. 31A-31G are cross-sectional side views of an optical bio-disc showing the steps of a method for performing an immunochemical assay using the nanoparticles of the present invention; and

FIGS. 32A-32G are enlarged partial cross-sectional side views of an optical bio-disc showing the steps of a method for performing a genetic assay using the nanoparticles of the present invention.

### DETAILED DESCRIPTION OF THE INVENTION

The present invention is directed in general to optical bio-disc assays, to synthesis of active micro-particles suitable for bio-disc assays, to disc drive systems, optical bio-discs, image processing techniques, counting methods, and related software. In particular, the invention relates to the synthesis of micro-particles for use in bio-disc assays and optical analysis discs adapted for use therewith. Each of the aspects of the present invention is discussed below in further detail.

Drive System and Related Discs

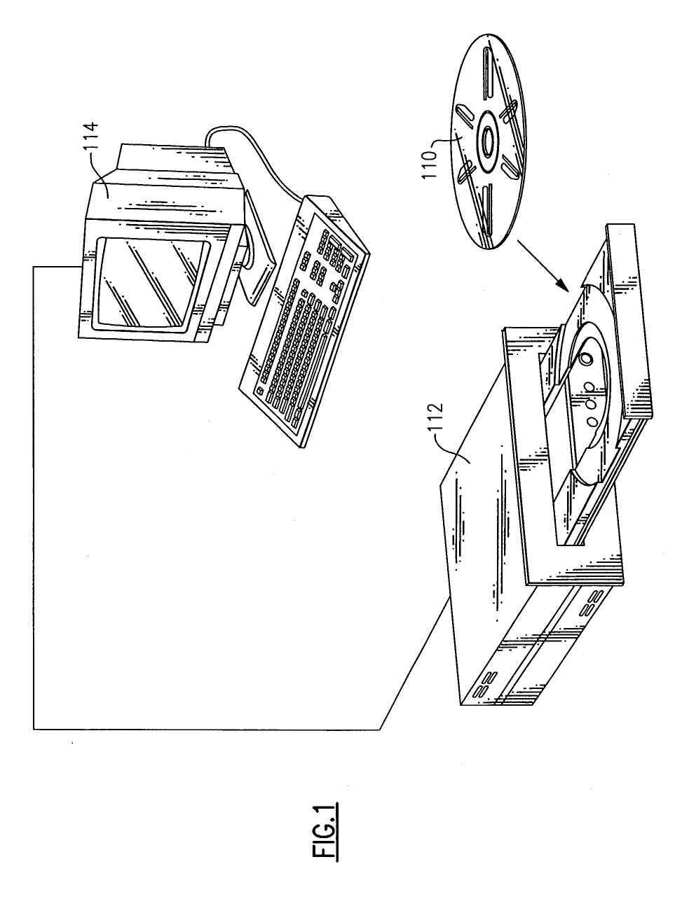

FIG. 1 is a perspective view of an optical bio-disc 110 according to the present invention as implemented to conduct the cell counts and differential cell counts disclosed herein. The present optical bio-disc 110 is shown in conjunction with an optical disc drive 112 and a display monitor 114. Further details relating to this type of disc drive and disc analysis system are disclosed in commonly assigned and co-pending U.S. patent application Ser. No. 10/008,156 entitled “Disc Drive System and Methods for Use with Bio-discs” filed Nov. 9, 2001 and U.S. patent application Ser. No. 10/043,688 entitled “Optical Disc Analysis System Including Related Methods For Biological and Medical Imaging” filed Jan. 10, 2002, both of which are herein incorporated by reference.

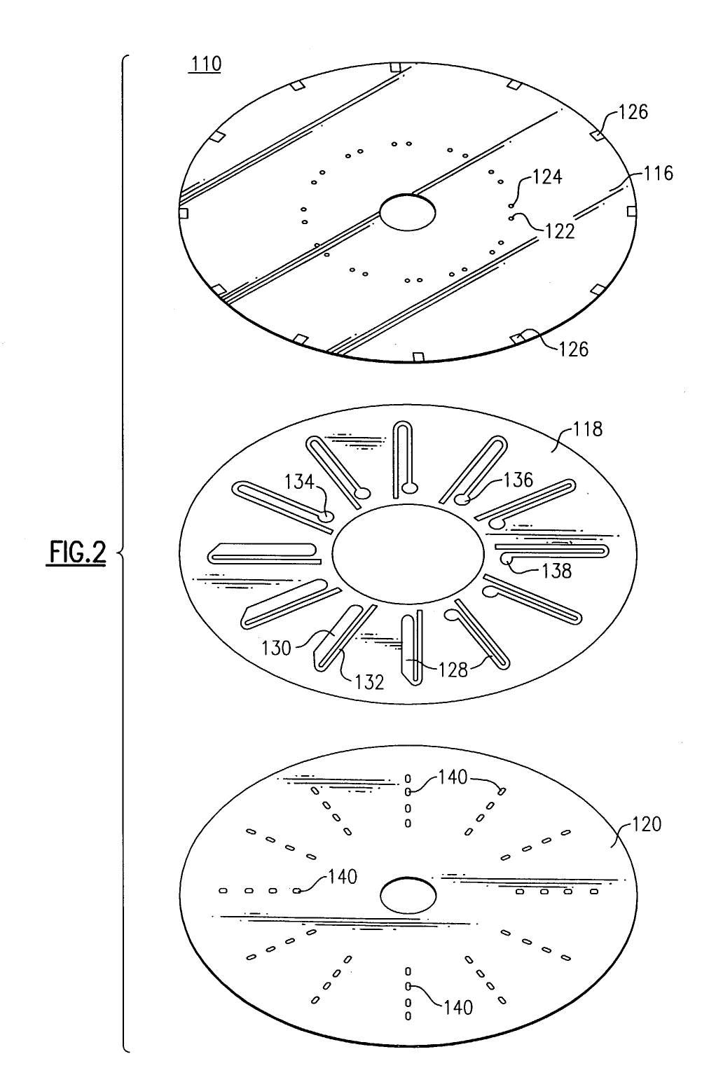

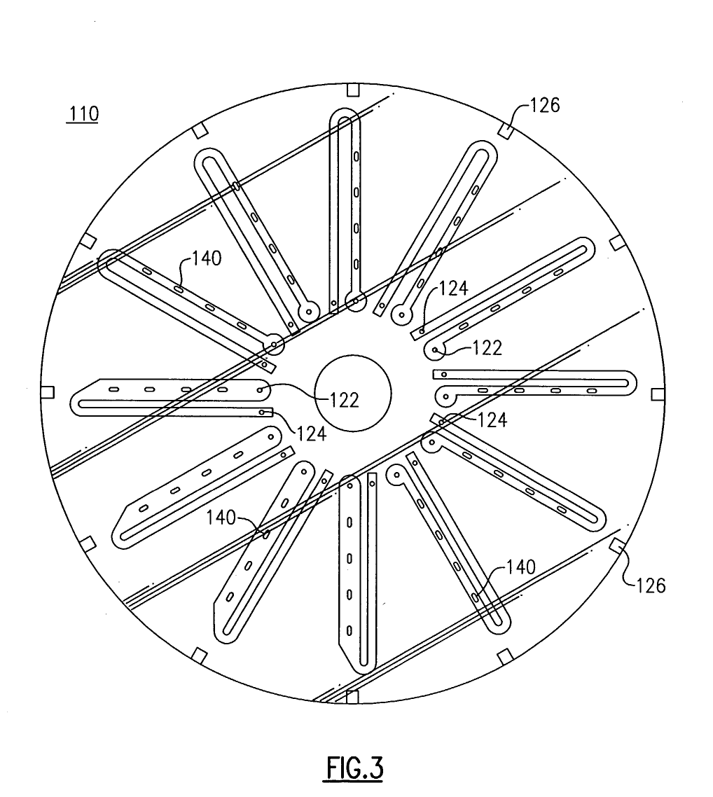

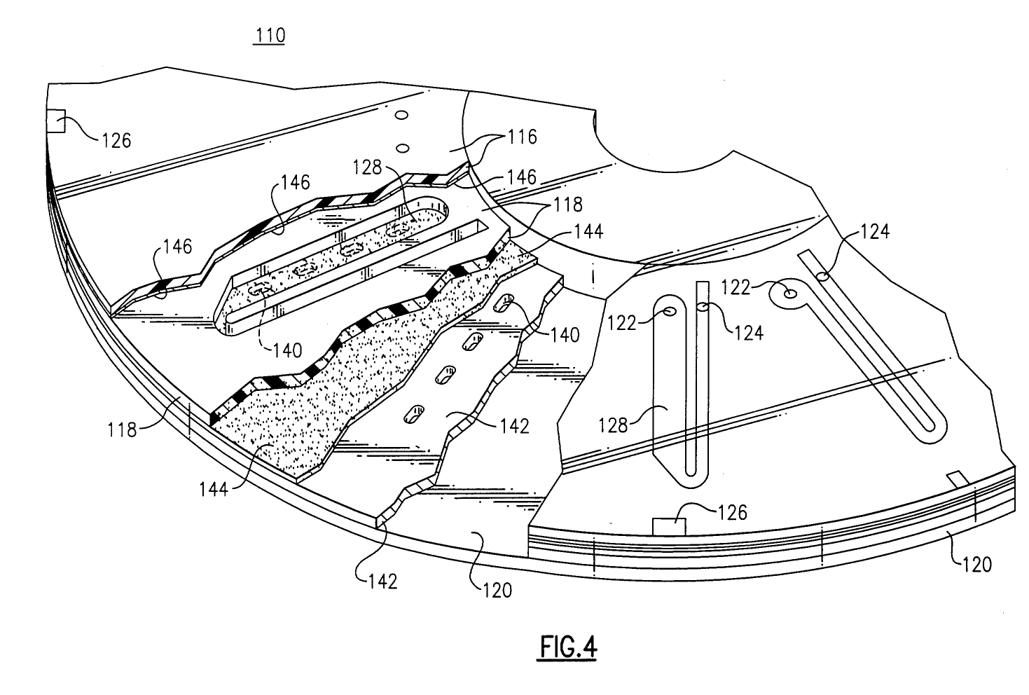

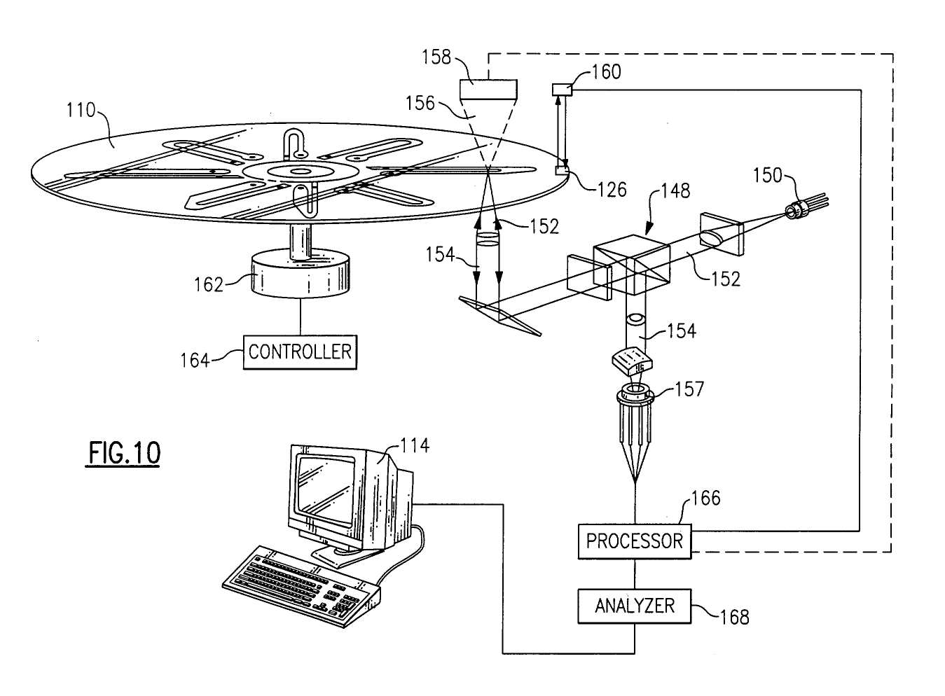

FIG. 2 is an exploded perspective view of the principal structural elements of one embodiment of the optical bio-disc 110. FIG. 2 is an example of a reflective zone optical bio-disc 110 (hereinafter “reflective disc”) that may be used in the present invention. The principal structural elements include a cap portion 116, an adhesive member or channel layer 118, and a substrate 120. The cap portion 116 includes one or more inlet ports 122 and one or more vent ports 124. The cap portion 116 may be formed from polycarbonate and is preferably coated with a reflective surface 146 (FIG. 4) on the bottom thereof as viewed from the perspective of FIG. 2. In the preferred embodiment, trigger marks or markings 126 are included on the surface of the reflective layer 142 (FIG. 4). Trigger markings 126 may include a clear window in all three layers of the bio-disc, an opaque area, or a reflective or semi-reflective area encoded with information that sends data to a processor 166, as shown FIG. 10, that in turn interacts with the operative functions of the interrogation or incident beam 152, FIGS. 6 and 10.

The second element shown in FIG. 2 is an adhesive member or channel layer 118 having fluidic circuits 128 or U-channels formed therein. The fluidic circuits 128 are formed by stamping or cutting the membrane to remove plastic film and form the shapes as indicated. Each of the fluidic circuits 128 includes a flow channel 130 and a return channel 132. Some of the fluidic circuits 128 illustrated in FIG. 2 include a mixing chamber 134. Two different types of mixing chambers 134 are illustrated. The first is a symmetric mixing chamber 136 that is symmetrically formed relative to the flow channel 130. The second is an off-set mixing chamber 138. The off-set mixing chamber 138 is formed to one side of the flow channel 130 as indicated.

The third element illustrated in FIG. 2 is a substrate 120 including target or capture zones 140. The substrate 120 is preferably made of polycarbonate and has a reflective layer 142 deposited on the top thereof, FIG. 4. The target zones 140 are formed by removing the reflective layer 142 in the indicated shape or alternatively in any desired shape. Alternatively, the target zone 140 may be formed by a masking technique that includes masking the target zone 140 area before applying the reflective layer 142. The reflective layer 142 may be formed from a metal such as aluminum or gold.

FIG. 3 is a top plan view of the optical bio-disc 110 illustrated in FIG. 2 with the reflective layer 142 on the cap portion 116 shown as transparent to reveal the fluidic circuits 128, the target zones 140, and trigger markings 126 situated within the disc.

FIG. 4 is an enlarged perspective view of the reflective zone type optical bio-disc 110 according to one embodiment of the present invention. This view includes a portion of the various layers thereof, cut away to illustrate a partial sectional view of each principal layer, substrate, coating, or membrane. FIG. 4 shows the substrate 120 that is coated with the reflective layer 142. An active layer 144 is applied over the reflective layer 142. In the preferred embodiment, the active layer 144 may be formed from polystyrene. Alternatively, polycarbonate, gold, activated glass, modified glass, or modified polystyrene, for example, polystyrene-co-maleic anhydride, may be used. In addition, hydrogels can be used. Alternatively as illustrated in this embodiment, the plastic adhesive member 118 is applied over the active layer 144. The exposed section of the plastic adhesive member 118 illustrates the cut out or stamped U-shaped form that creates the fluidic circuits 128. The final principal structural layer in this reflective zone embodiment of the present bio-disc is the cap portion 116. The cap portion 116 includes the reflective surface 146 on the bottom thereof. The reflective surface 146 may be made from a metal such as aluminum or gold.

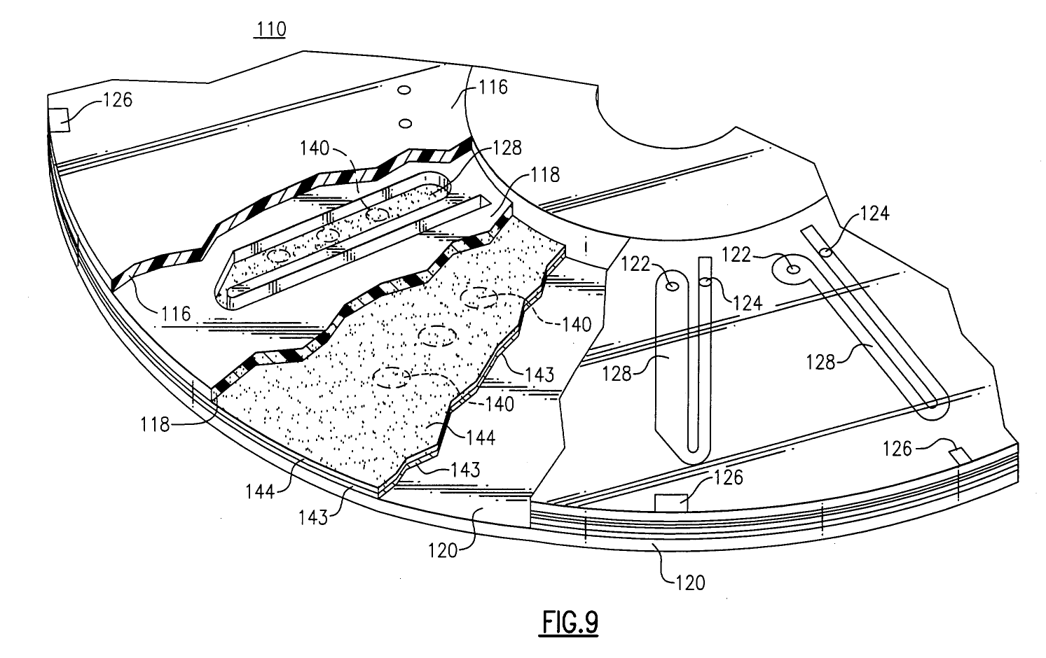

Referring now to FIG. 5, there is shown an exploded perspective view of the principal structural elements of a transmissive type of optical bio-disc 110 according to the present invention. The principal structural elements of the transmissive type of optical bio-disc 110 similarly include the cap portion 116, the adhesive or channel member 118, and the substrate 120 layer. The cap portion 116 includes one or more inlet ports 122 and one or more vent ports 124. The cap portion 116 may be formed from a polycarbonate layer. Optional trigger markings 126 may be included on the surface of a thin semi-reflective layer 143, as best illustrated in FIGS. 6 and 9. Trigger markings 126 may include a clear window in all three layers of the bio-disc, an opaque area, or a reflective or semi-reflective area encoded with information that sends data to the processor 166, FIG. 10, which in turn interacts with the operative functions of the interrogation beam 152, FIGS. 6 and 10.

The second element shown in FIG. 5 is the adhesive member or channel layer 118 having fluidic circuits 128 or U-channels formed therein. The fluidic circuits 128 are formed by stamping or cutting the membrane to remove plastic film and form the shapes as indicated. Each of the fluidic circuits 128 includes the flow channel 130 and the return channel 132. Some of the fluidic circuits 128 illustrated in FIG. 5 include the mixing chamber 134. Two different types of mixing chambers 134 are illustrated. The first is the symmetric mixing chamber 136 that is symmetrically formed relative to the flow channel 130. The second is the off-set mixing chamber 138. The off-set mixing chamber 138 is formed to one side of the flow channel 130 as indicated.

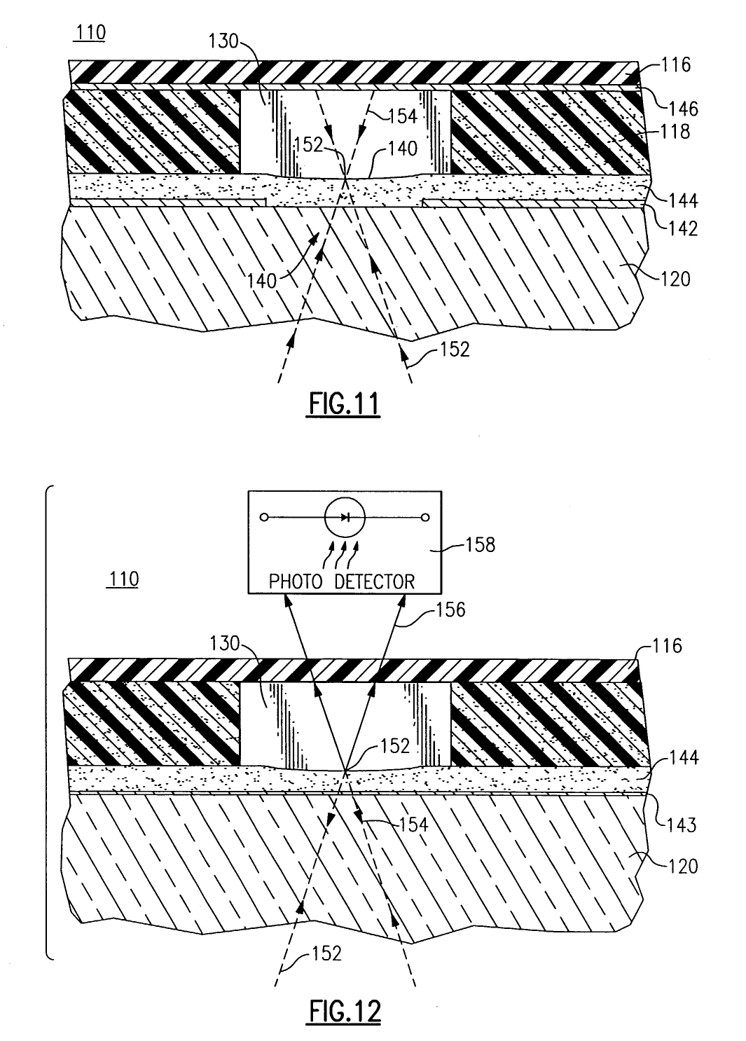

The third element illustrated in FIG. 5 is the substrate 120, which may include the target or capture zones 140. The substrate 120 is preferably made of polycarbonate and has the thin semi-reflective layer 143 deposited on the top thereof, FIG. 6. The semi-reflective layer 143 associated with the substrate 120 of the disc 110 illustrated in FIGS. 5 and 6 is significantly thinner than the reflective layer 142 on the substrate 120 of the reflective disc 110 illustrated in FIGS. 2, 3 and 4. The thinner semi-reflective layer 143 allows for some transmission of the interrogation beam 152 through the structural layers of the transmissive disc as shown in FIGS. 6 and 12. The thin semi-reflective layer 143 may be formed from a metal such as aluminum or gold.

FIG. 6 is an enlarged perspective view of the substrate 120 and semi-reflective layer 143 of the transmissive embodiment of the optical bio-disc 110 illustrated in FIG. 5. The thin semi-reflective layer 143 may be made from a metal such as aluminum or gold. In the preferred embodiment, the thin semi-reflective layer 143 of the transmissive disc illustrated in FIGS. 5 and 6 is approximately 100-300 Å thick and does not exceed 400 Å. This thinner semi-reflective layer 143 allows a portion of the incident or interrogation beam 152 to penetrate and pass through the semi-reflective layer 143 to be detected by a top detector 158, FIGS. 10 and 12, while some of the light is reflected or returned back along the incident path. As indicated below, Table 1 presents the reflective and transmissive characteristics of a gold film relative to the thickness of the film. The gold film layer is fully reflective at a thickness greater than 800 Å. While the threshold density for transmission of light through the gold film is approximately 400 Å.

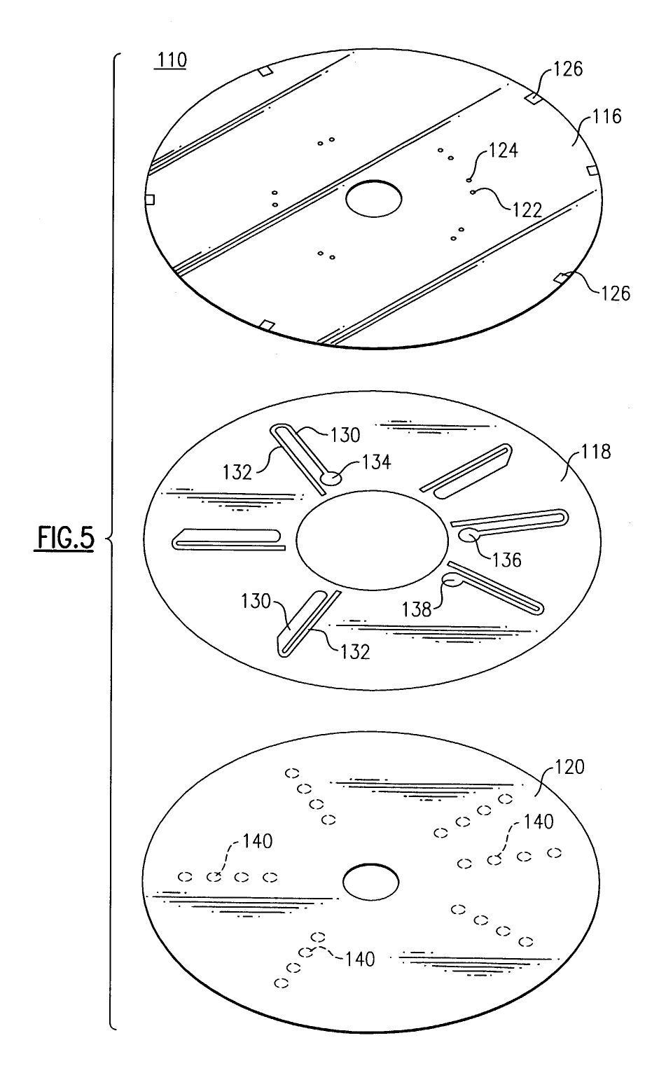

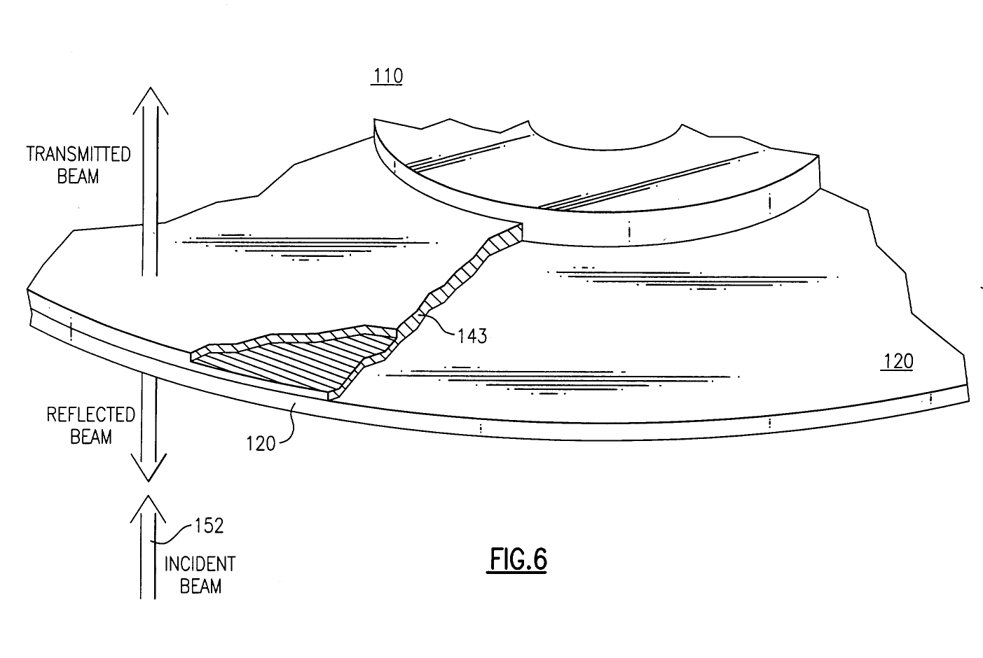

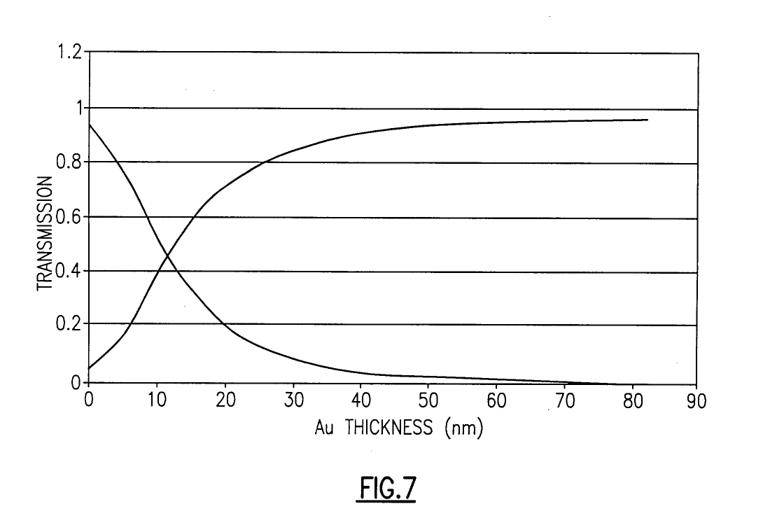

In addition to Table 1, FIG. 7 provides a graphical representation of the inverse relationship of the reflective and transmissive nature of the thin semi-reflective layer 143 based upon the thickness of the gold. Reflective and transmissive values used in the graph illustrated in FIG. 7 are absolute values.

TABLE 1
Au Film Reflection and Transmission (Absolute Values)

| Thickness | Thickness | | |
|---|---|---|---|
| (Angstroms) | (nm) | Reflectance | Transmittance |
| 0 | 0 | 0.0505 | 0.9495 |
| 50 | 5 | 0.1683 | 0.7709 |
| 100 | 10 | 0.3981 | 0.5169 |
| 150 | 15 | 0.5873 | 0.3264 |
| 200 | 20 | 0.7142 | 0.2057 |
| 250 | 25 | 0.7959 | 0.1314 |
| 300 | 30 | 0.8488 | 0.0851 |
| 350 | 35 | 0.8836 | 0.0557 |
| 400 | 40 | 0.9067 | 0.0368 |
| 450 | 45 | 0.9222 | 0.0244 |
| 500 | 50 | 0.9328 | 0.0163 |
| 550 | 55 | 0.9399 | 0.0109 |
| 600 | 60 | 0.9448 | 0.0073 |
| 650 | 65 | 0.9482 | 0.0049 |
| 700 | 70 | 0.9505 | 0.0033 |
| 750 | 75 | 0.9520 | 0.0022 |
| 800 | 80 | 0.9531 | 0.0015 |

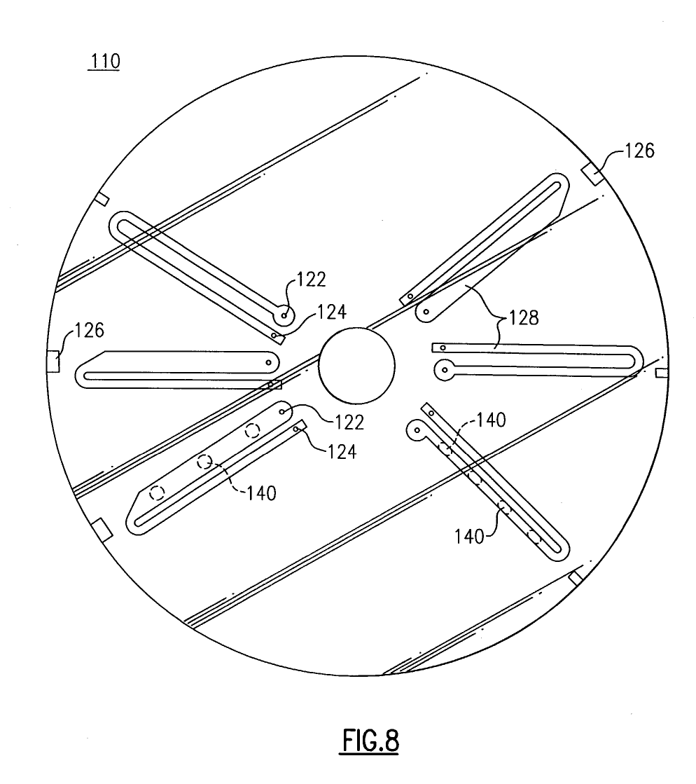

With reference next to FIG. 8, there is shown a top plan view of the transmissive type optical bio-disc 110 illustrated in FIGS. 5 and 6 with the transparent cap portion 116 revealing the fluidic channels, the trigger markings 126, and the target zones 140 as situated within the disc.

FIG. 9 is an enlarged perspective view of the optical bio-disc 110 according to the transmissive disc embodiment of the present invention. The disc 110 is illustrated with a portion of the various layers thereof cut away to show a partial sectional view of each principal layer, substrate, coating, or membrane. FIG. 9 illustrates a transmissive disc format with the clear cap portion 116, the thin semi-reflective layer 143 on the substrate 120, and trigger markings 126. In this embodiment, trigger markings 126 include opaque material placed on the top portion of the cap. Alternatively the trigger marking 126 may be formed by clear, non-reflective windows etched on the thin reflective layer 143 of the disc, or any mark that absorbs or does not reflect the signal coming from the trigger detector 160, FIG. 10. FIG. 9 also shows, the target zones 140 formed by marking the designated area in the indicated shape or alternatively in any desired shape. Markings to indicate target zone 140 may be made on the thin semi-reflective layer 143 on the substrate 120 or on the bottom portion of the substrate 120 (under the disc). Alternatively, the target zones 140 may be formed by a masking technique that includes masking the entire thin semi-reflective layer 143 except the target zones 140. In this embodiment, target zones 140 may be created by silk screening ink onto the thin semi-reflective layer 143. In the transmissive disc format illustrated in FIGS. 5, 8, and 9, the target zones 140 may alternatively be defined by address information encoded on the disc. In this embodiment, target zones 140 do not include a physically discernable edge boundary.

With continuing reference to FIG. 9, an active layer 144 is illustrated as applied over the thin semi-reflective layer 143. In the preferred embodiment, the active layer 144 is a 10 to 200 μm thick layer of 2% polystyrene. Alternatively, polycarbonate, gold, activated glass, modified glass, or modified polystyrene, for example, polystyrene-co-maleic anhydride, may be used. In addition, hydrogels can be used. As illustrated in this embodiment, the plastic adhesive member 118 is applied over the active layer 144. The exposed section of the plastic adhesive member 118 illustrates the cut out or stamped U-shaped form that creates the fluidic circuits 128.

The final principal structural layer in this transmissive embodiment of the present bio-disc 110 is the clear, non-reflective cap portion 116 that includes inlet ports 122 and vent ports 124.

Referring now to FIG. 10, there is a representation in perspective and block diagram illustrating optical components 148, a light source 150 that produces the incident or interrogation beam 152, a return beam 154, and a transmitted beam 156. In the case of the reflective bio-disc illustrated in FIG. 4, the return beam 154 is reflected from the reflective surface 146 of the cap portion 116 of the optical bio-disc 110. In this reflective embodiment of the present optical bio-disc 110, the return beam 154 is detected and analyzed for the presence of signal elements by a bottom detector 157. In the transmissive bio-disc format, on the other hand, the transmitted beam 156 is detected, by a top detector 158, and is also analyzed for the presence of signal elements. In the transmissive embodiment, a photo detector may be used as a top detector 158.

FIG. 10 also shows a hardware trigger mechanism that includes the trigger markings 126 on the disc and a trigger detector 160. The hardware triggering mechanism is used in both reflective bio-discs (FIG. 4) and transmissive bio-discs (FIG. 9). The triggering mechanism allows the processor 166 to collect data only when the interrogation beam 152 is on a respective target zone 140. Furthermore, in the transmissive bio-disc system, a software trigger may also be used. The software trigger uses the bottom detector to signal the processor 166 to collect data as soon as the interrogation beam 152 hits the edge of a respective target zone 140. FIG. 10 further illustrates a drive motor 162 and a controller 164 for controlling the rotation of the optical bio-disc 110. FIG. 10 also shows the processor 166 and analyzer 168 implemented in the alternative for processing the return beam 154 and transmitted beam 156 associated the transmissive optical bio-disc.

As shown in FIG. 11, there is presented a partial cross sectional view of the reflective disc embodiment of the optical bio-disc 110 according to the present invention. FIG. 11 illustrates the substrate 120 and the reflective layer 142. As indicated above, the reflective layer 142 may be made from a material such as aluminum, gold or other suitable reflective material. In this embodiment, the top surface of the substrate 120 is smooth. FIG. 11 also shows the active layer 144 applied over the reflective layer 142. As also shown in FIG. 11, the target zone 140 is formed by removing an area or portion of the reflective layer 142 at a desired location or, alternatively, by masking the desired area prior to applying the reflective layer 142. As further illustrated in FIG. 11, the plastic adhesive member 118 is applied over the active layer 144. FIG. 11 also shows the cap portion 116 and the reflective surface 146 associated therewith. Thus when the cap portion 116 is applied to the plastic adhesive member 118 including the desired cutout shapes, flow channel 130 is thereby formed. As indicated by the arrowheads shown in FIG. 11, the path of the incident beam 152 is initially directed toward the substrate 120 from below the disc 110. The incident beam then focuses at a point proximate the reflective layer 142. Since this focusing takes place in the target zone 140 where a portion of the reflective layer 142 is absent, the incident continues along a path through the active layer 144 and into the flow channel 130. The incident beam 152 then continues upwardly traversing through the flow channel to eventually fall incident onto the reflective surface 146. At this point, the incident beam 152 is returned or reflected back along the incident path and thereby forms the return, beam 154.

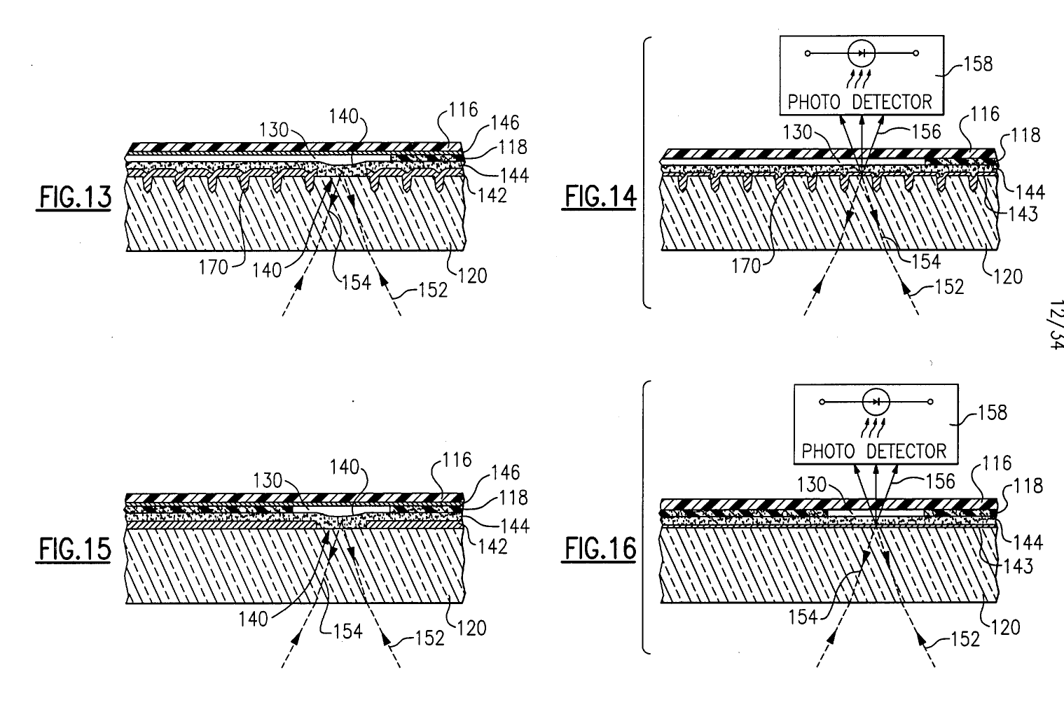

FIG. 12 is a partial cross sectional view of the transmissive embodiment of the bio-disc 110 according to the present invention. FIG. 12 illustrates a transmissive disc format with the clear cap portion 116 and the thin semi-reflective layer 143 on the substrate 120. FIG. 12 also shows the active layer 144 applied over the thin semi-reflective layer 143. In the preferred embodiment, the transmissive disc has the thin semi-reflective layer 143 made from a metal such as aluminum or gold approximately 100-300 Angstroms thick and does not exceed 400 Angstroms. This thin semi-reflective layer 143 allows a portion of the incident or interrogation beam 152, from the light source 150, FIG. 10, to penetrate and pass upwardly through the disc to be detected by a top detector 158, while some of the light is reflected back along the same path as the incident beam but in the opposite direction. In this arrangement, the return or reflected beam 154 is reflected from the semi-reflective layer 143. Thus in this manner, the return beam 154 does not enter into the flow channel 130. The reflected light or return beam 154 may be used for tracking the incident beam 152 on pre-recorded information tracks formed in or on the semi-reflective layer 143 as described in more detail in conjunction with FIGS. 13 and 14. In the disc embodiment illustrated in FIG. 12, a physically defined target zone 140 may or may not be present. Target zone 140 may be created by direct markings made on the thin semi-reflective layer 143 on the substrate 120. These marking may be formed using silk screening or any equivalent method. In the alternative embodiment where no physical indicia are employed to define a target zone (such as, for example, when encoded software addressing is utilized) the flow channel 130 in effect may be employed as a confined target area in which inspection of an investigational feature is conducted.

FIG. 13 is a cross sectional view taken across the tracks of the reflective disc embodiment of the bio-disc 110 according to the present invention. This view is taken longitudinally along a radius and flow channel of the disc. FIG. 13 includes the substrate 120 and the reflective layer 142. In this embodiment, the substrate 120 includes a series of grooves 170. The grooves 170 are in the form of a spiral extending from near the center of the disc toward the outer edge. The grooves 170 are implemented so that the interrogation beam 152 may track along the spiral grooves 170 on the disc. This type of groove 170 is known as a “wobble groove”. A bottom portion having undulating or wavy sidewalls forms the groove 170, while a raised or elevated portion separates adjacent grooves 170 in the spiral. The reflective layer 142 applied over the grooves 170 in this embodiment is, as illustrated, conformal in nature. FIG. 13 also shows the active layer 144 applied over the reflective layer 142. As shown in FIG. 13, the target zone 140 is formed by removing an area or portion of the reflective layer 142 at a desired location or, alternatively, by masking the desired area prior to applying the reflective layer 142. As further illustrated in FIG. 13, the plastic adhesive member 118 is applied over the active layer 144. FIG. 13 also shows the cap portion 116 and the reflective surface 146 associated therewith. Thus, when the cap portion 116 is applied to the plastic adhesive member 118 including the desired cutout shapes, the flow channel 130 is thereby formed.

FIG. 14 is a cross sectional view taken across the tracks of the transmissive disc embodiment of the bio-disc 110 according to the present invention as described in FIG. 12, for example. This view is taken longitudinally along a radius and flow channel of the disc. FIG. 14 illustrates the substrate 120 and the thin semi-reflective layer 143. This thin semi-reflective layer 143 allows the incident or interrogation beam 152, from the light source 150, to penetrate and pass through the disc to be detected by the top detector 158, while some of the light is reflected back in the form of the return beam 154. The thickness of the thin semi-reflective layer 143 is determined by the minimum amount of reflected light required by the disc reader to maintain its tracking ability. The substrate 120 in this embodiment, like that discussed in FIG. 13, includes the series of grooves 170. The grooves 170 in this embodiment are also preferably in the form of a spiral extending from near the center of the disc toward the outer edge. The grooves 170 are implemented so that the interrogation beam 152 may track along the spiral. FIG. 14 also shows the active layer 144 applied over the thin semi-reflective layer 143. As further illustrated in FIG. 14, the plastic adhesive member or channel layer 118 is applied over the active layer 144. FIG. 14 also shows the cap portion 116 without a reflective surface 146. Thus, when the cap is applied to the plastic adhesive member 118 including the desired cutout shapes, the flow channel 130 is thereby formed and a part of the incident beam 152 is allowed to pass therethrough substantially unreflected.

FIG. 15 is a view similar to FIG. 11 showing the entire thickness of the reflective disc and the initial refractive property thereof. FIG. 16 is a view similar to FIG. 12 showing the entire thickness of the transmissive disc and the initial refractive property thereof. Grooves 170 are not seen in FIGS. 15 and 16 since the sections are cut along the grooves 170. FIGS. 15 and 16 show the presence of the narrow flow channel 130 that is situated perpendicular to the grooves 170 in these embodiments. FIGS. 13, 14, 15, and 16 show the entire thickness of the respective reflective and transmissive discs. In these figures, the incident beam 152 is illustrated initially interacting with the substrate 120 which has refractive properties that change the path of the incident beam as illustrated to provide focusing of the beam 152 on the reflective layer 142 or the thin semi-reflective layer 143.

Counting Methods and Related Software

By way of illustrative background, a number of methods and related algorithms for white blood cell counting using optical disc data are herein discussed in further detail. These methods and related algorithms are not limited to counting white blood cells, but may be readily applied to conducting counts of any type of particulate matter including, but not limited to, red blood cells, white blood cells, beads, microparticles and any other objects, both biological and non-biological, that produce similar optical signatures that can be detected by an optical reader.

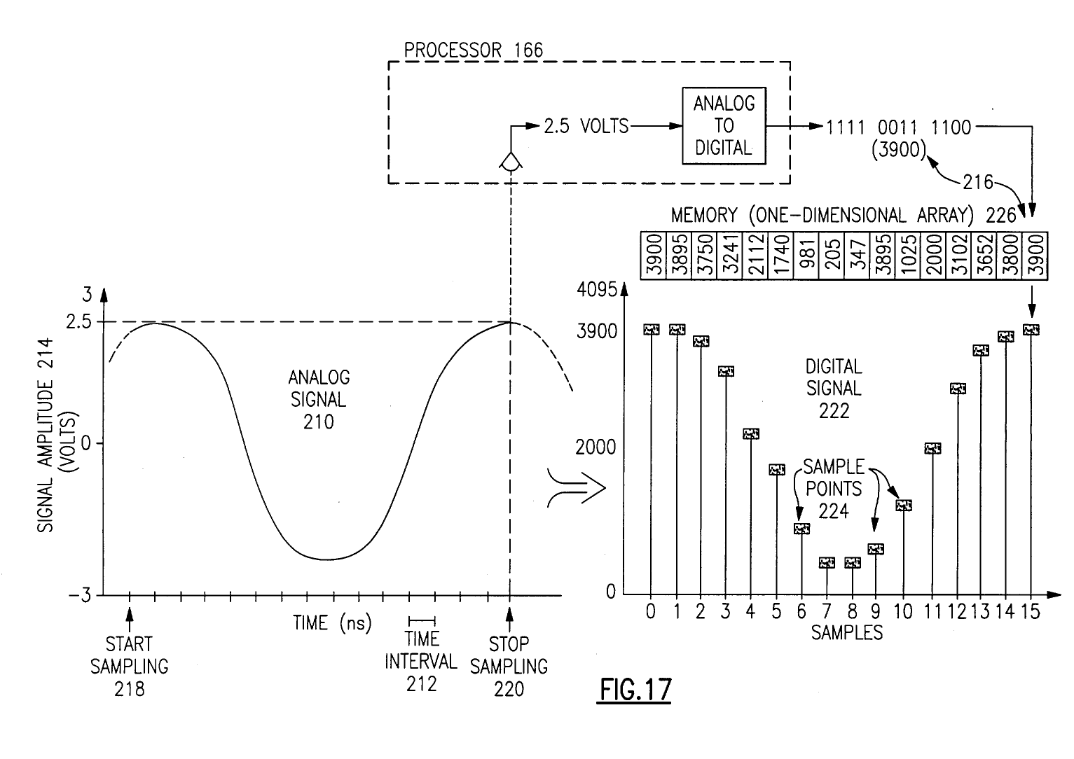

For the purposes of illustration, the following description of the methods and algorithms related to the present invention as described with reference to FIGS. 17-21, are directed to cell counting. With some modifications, these methods and algorithms can be applied to counting the synthetic microparticles, nanocapsules, or nanoparticles of the present invention. The data evaluation aspects of the cell counting methods and algorithms are described generally herein to provide related background for the methods and apparatus of the present invention. Methods and algorithms for capturing and processing investigational data from the optical bio-disc has general broad applicability and has been disclosed in further detail in commonly assigned U.S. Provisional Application No. 60/291,233 entitled “Variable Sampling Control For Rendering Pixelation of Analysis Results In Optical Bio-Disc Assembly And Apparatus Relating Thereto” filed May 16, 2001 which is herein incorporated by reference and the above incorporated U.S. Provisional Application No. 60/404,921 entitled “Methods For Differential Cell Counts Including Related Apparatus And Software For Performing Same”. In the following discussion, the basic scheme of the methods and algorithms with a brief explanation is presented. As illustrated in FIG. 10, information concerning attributes of the biological test sample is retrieved from the optical bio-disc 110 in the form of a beam of electromagnetic radiation that has been modified or modulated by interaction with the test sample. In the case of the reflective optical bio-disc discussed in conjunction with FIGS. 2, 3, 4, 11, 13, and 15, the return beam 154 carries the information about the biological sample. As discussed above, such information about the biological sample is contained in the return beam essentially only when the incident beam is within the flow channel 130 or target zones 140 and thus in contact with the sample. In the reflective embodiment of the bio-disc 110, the return beam 154 may also carry information encoded in or on the reflective layer 142 or otherwise encoded in the wobble grooves 170 illustrated in FIGS. 13 and 14. As would be apparent to one of skill in the art, pre-recorded information is contained in the return beam 154 of the reflective disc with target zones, only when the corresponding incident beam is in contact with the reflective layer 142. Such information is not contained in the return beam 154 when the incident beam 152 is in an area where the information bearing reflective layer 142 has been removed or is otherwise absent. In the case of the transmissive optical bio-disc discussed in conjunction with FIGS. 5, 6, 8, 9, 12, 14, and 16, the transmitted beam 156 carries the information about the biological sample.

With continuing reference to FIG. 10, the information about the biological test sample, whether it is obtained from the return beam 154 of the reflective disc or the transmitted beam 156 of the transmissive disc, is directed to a processor 166 for signal processing. This processing involves transformation of the analog signal detected by the bottom detector 157 (reflective disc) or the top detector 158 (transmissive disc) to a discrete digital form.

Referring next to FIG. 17, the signal transformation involves sampling the analog signal 210 at fixed time intervals 212, and encoding the corresponding instantaneous analog amplitude 214 of the signal as a discrete binary integer 216. Sampling is started at some start time 218 and stopped at some end time 220. The two common values associated with any analog-to-digital conversion process are sampling frequency and bit depth. The sampling frequency, also called the sampling rate, is the number of samples taken per unit time. A higher sampling frequency yields a smaller time interval 212 between consecutive samples, which results in a higher fidelity of the digital signal 222 compared to the original analog signal 210. Bit depth is the number of bits used in each sample point to encode the sampled amplitude 214 of the analog signal 210. The greater the bit depth, the better the binary integer 216 will approximate the original analog amplitude 214. In the present embodiment, the sampling rate is 8 MHz with a bit depth of 12 bits per sample, allowing an integer sample range of 0 to 4095 (0 to (2ⁿ−1), where n is the bit depth. This combination may change to accommodate the particular accuracy necessary in other embodiments. By way of example and not limitation, it may be desirable to increase sampling frequency in embodiments involving methods for counting beads, which are generally smaller than cells. The sampled data is then sent to processor 166 for analog-to-digital transformation.

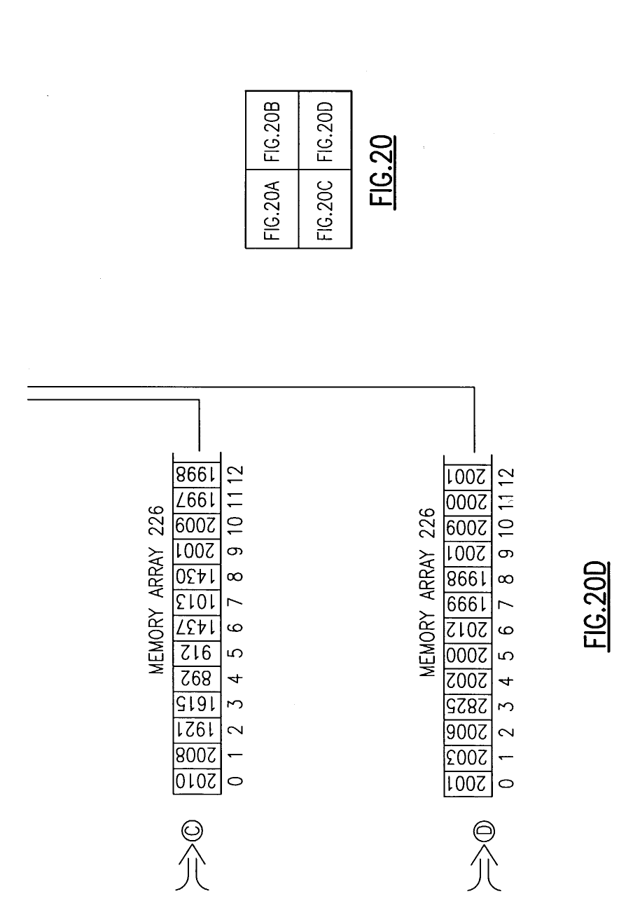

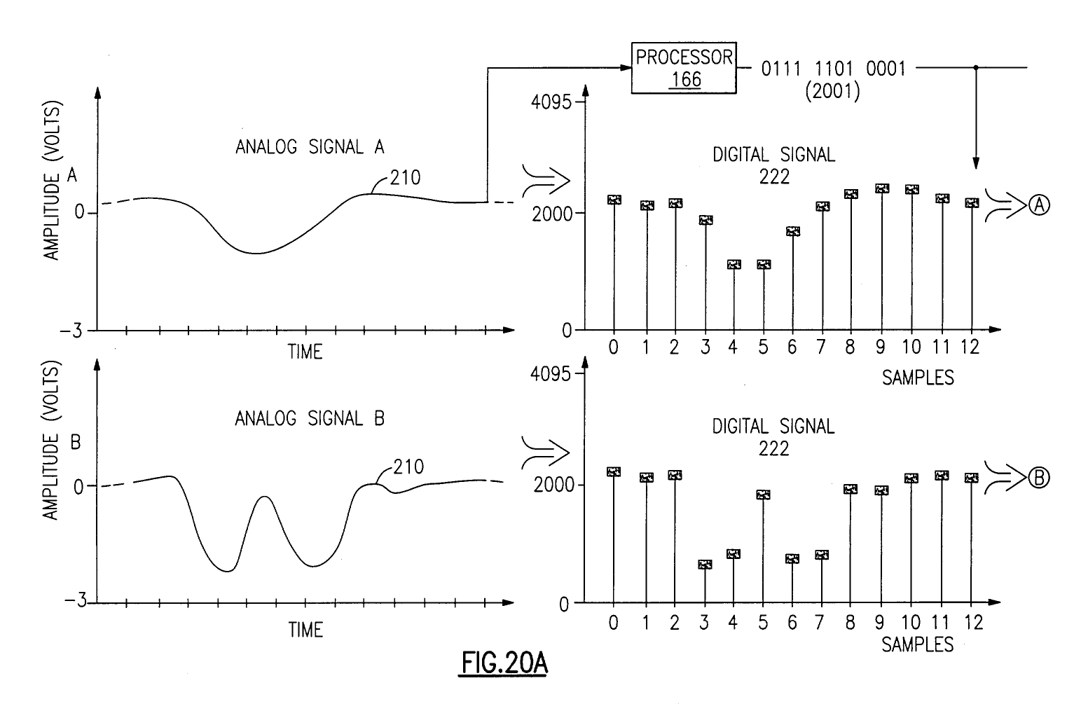

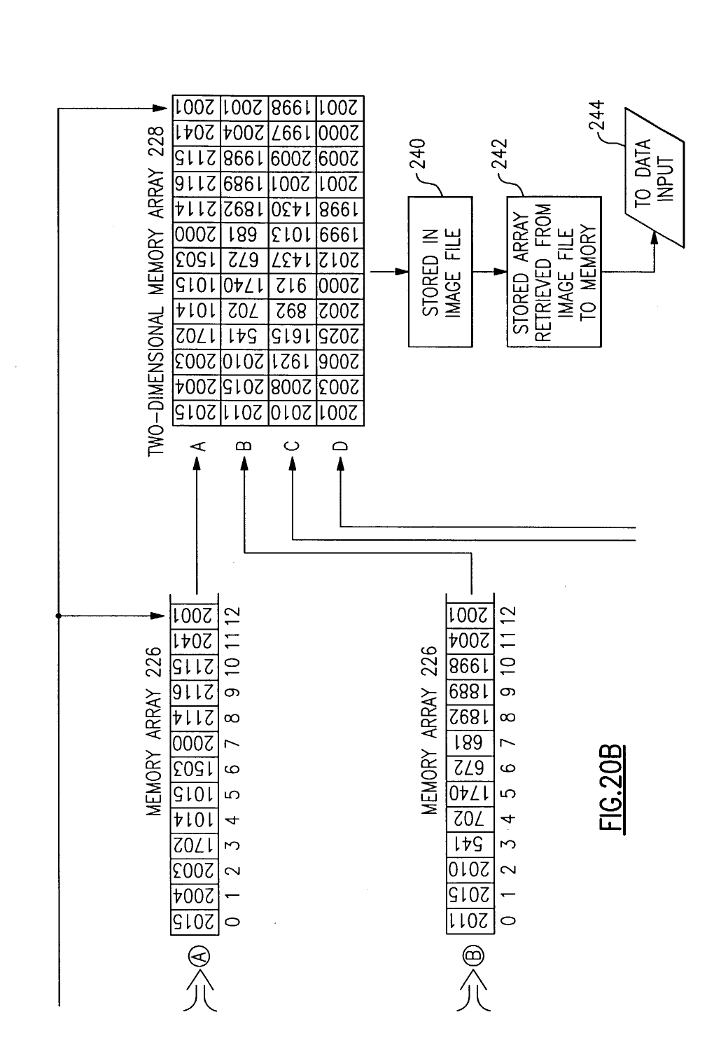

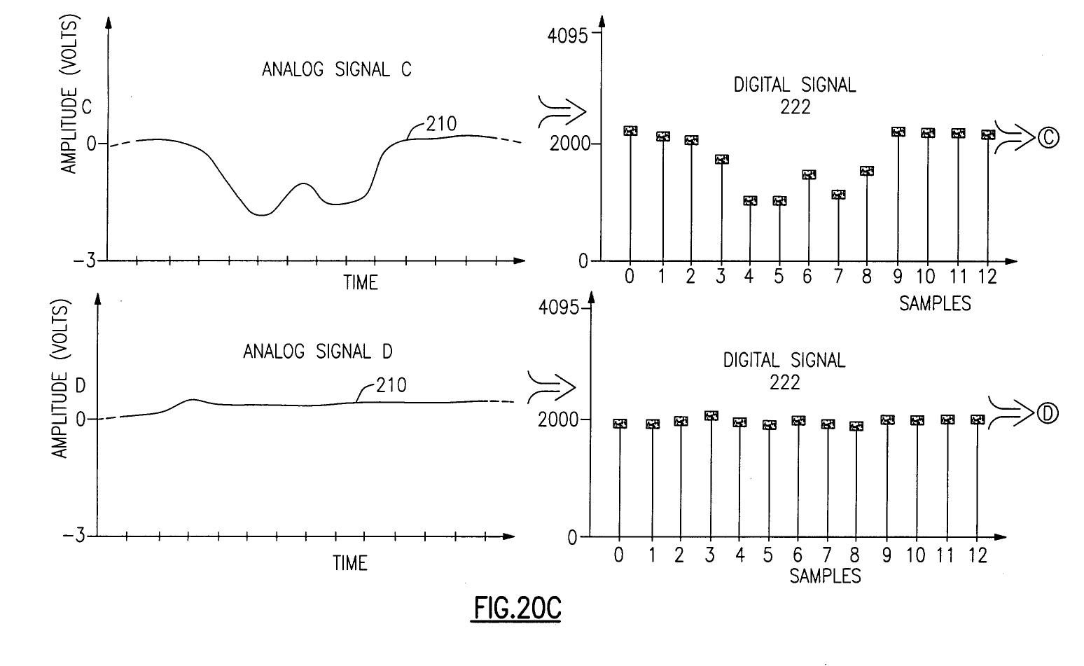

During the analog-to-digital transformation, each consecutive sample point 224 along the laser path is stored consecutively on disc or in memory as a one-dimensional array 226. Each consecutive track contributes an independent one-dimensional array, which yields a two-dimensional array 228 (FIG. 20A) that is analogous to an image.

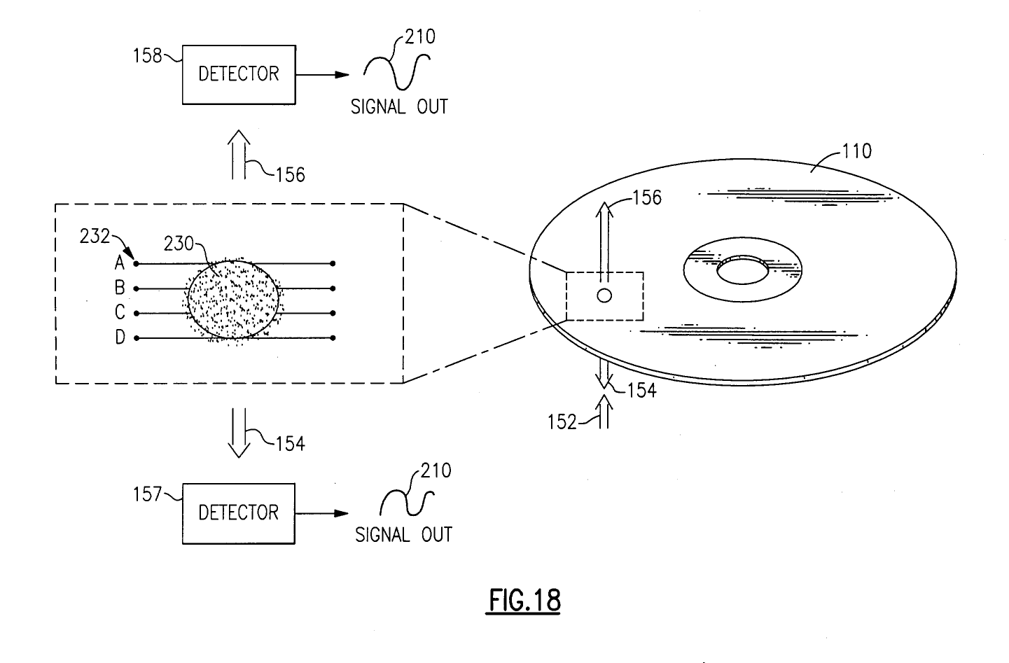

FIG. 18 is a perspective view of an optical bio-disc 110 of the present invention with an enlarged detailed perspective view of the section indicated showing a captured white blood cell 230 positioned relative to the tracks 232 of the optical bio-disc. The white blood cell 230 is used herein for illustrative purposes only. As indicated above, other objects or investigational features such as beads or agglutinated matter may be utilized herewith. As shown, the interaction of incident beam 152 with white blood cell 230 yields a signal-containing beam, either in the form of a return beam 154 of the reflective disc or a transmitted beam 156 of the transmissive disc, which is detected by either of detectors 157 or 158.

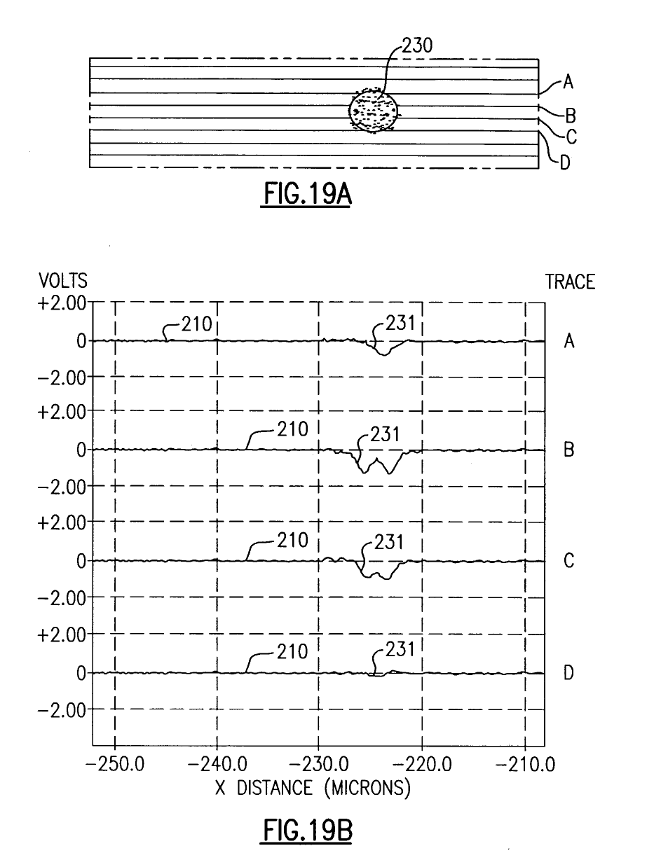

FIG. 19A is another graphical representation of the white blood cell 230 positioned relative to the tracks 232 of the optical bio-disc 110 shown in FIG. 18. As shown in FIGS. 18 and 19A, the white blood cell 230 covers approximately four tracks A, B, C, and D. FIG. 19B shows a series of signature traces derived from the white blood cell 210 of FIGS. 19 and 19A. As indicated in FIG. 19B, the detection system provides four analogue signals A, B, C, and D corresponding to tracks A, B, C, and D. As further shown in FIG. 19B, each of the analogue signals A, B, C, and D carries specific information about the white blood cell 230. Thus as illustrated, a scan over a white blood cell 230 yields distinct perturbations of the incident beam that can be detected and processed. The analog signature traces (signals) 210 are then directed to processor 166 for transformation to an analogous digital signal 222 as shown in FIGS. 20A and 20C as discussed in further detail below.

FIG. 20 is a graphical representation illustrating the relationship between FIGS. 20A, 20B, 20C, and 20D. FIGS. 20A, 20B, 20C, and 20D are pictorial graphical representations of transformation of the signature traces from FIG. 19B into digital signals 222 that are stored as one-dimensional arrays 226 and combined into a two-dimensional array 228 for data input 244.

With particular reference now to FIG. 20A, there is shown sampled analog signals 210 from tracks A and B of the optical bio-disc shown in FIGS. 18 and 19A. Processor 166 then encodes the corresponding instantaneous analog amplitude 214 of the analog signal 210 as a discrete binary integer 216 (see FIG. 17). The resulting series of data points is the digital signal 222 that is analogous to the sampled analog signal 210.

Referring next to FIG. 20B, digital signal 222 from tracks A and B (FIG. 20A) is stored as an independent one-dimensional memory array 226. Each consecutive track contributes a corresponding one-dimensional array, which when combined with the previous one-dimensional arrays, yields a two-dimensional array 228 that is analogous to an image. The digital data is then stored in memory or on disc as a two-dimensional array 228 of sample points 224 (FIG. 17) that represent the relative intensity of the return beam 154 or transmitted beam 156 (FIG. 18) at a particular point in the sample area. The two-dimensional array is then stored in memory or on disc in the form of a raw file or image file 240 as represented in FIG. 20B. The data stored in the image file 240 is then retrieved 242 to memory and used as data input 244 to analyzer 168 shown in FIG. 10.

FIG. 20C shows sampled analog signals 210 from tracks C and D of the optical bio-disc shown in FIGS. 18 and 19A. Processor 166 then encodes the corresponding instantaneous analog amplitude 214 of the analog signal 210 as a discrete binary integer 216 (FIG. 17). The resulting series of data points is the digital signal 222 that is analogous to the sampled analog signal 210.

Referring now to FIG. 20D, digital signal 222 from tracks C and D is stored as an independent one-dimensional memory array 226. Each consecutive track contributes a corresponding one-dimensional array, which when combined with the previous one-dimensional arrays, yields a two-dimensional array 228 that is analogous to an image. As above, the digital data is then stored in memory or on disc as a two-dimensional array 228 of sample points 224 (FIG. 17) that represent the relative intensity of the return beam 154 or transmitted beam 156 (FIG. 18) at a particular point in the sample area. The two-dimensional array is then stored in memory or on disc in the form of a raw file or image file 240 as shown in FIG. 20B. As indicated above, the data stored in the image file 240 is then retrieved 242 to memory and used as data input 244 to analyzer 168 FIG. 10.

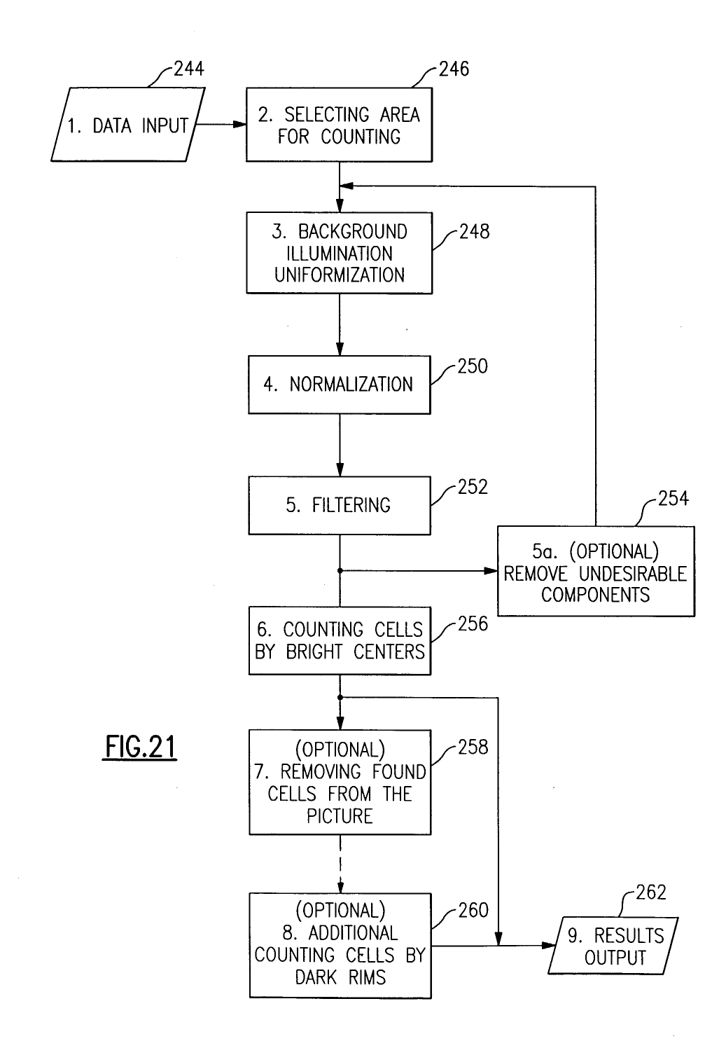

The computational and processing algorithms of the present invention are stored in analyzer 168 (FIG. 10) and applied to the input data 244 to produce useful output results 262 (FIG. 21) that may be displayed on the display monitor 114 (FIG. 10).

With reference now to FIG. 21 there is shown a logic flow chart of the principal steps for data evaluation according to the processing methods and computational algorithms related to the present invention. A first principal step of the present processing method involves receipt of the input data 244. As described above, data evaluation starts with an array of integers in the range of 0 to 4096.

The next principle step 246 is selecting an area of the disc for counting. Once this area is defined, an objective then becomes making an actual count of all white blood cells contained in the defined area. The implementation of step 246 depends on the configuration of the disc and user's options. By way of example and not limitation, embodiments of the invention using discs with windows such as the target zones 140 shown in FIGS. 2 and 5, the software recognizes the windows and crops a section thereof for analysis and counting. In one preferred embodiment, such as that illustrated in FIG. 2, the target zones or windows have the shape of 1×2 mm rectangles with a semicircular section on each end thereof. In this embodiment, the software crops a standard rectangle of 1×2 mm area inside a respective window. In an aspect of this embodiment, the reader may take several consecutive sample values to compare the number of cells in several different windows.

In embodiments of the invention using a transmissive disc without windows, as shown in FIGS. 5, 6, 8, and 9, step 246 may be performed in one of two different manners. The position of the standard rectangle is chosen either by positioning its center relative to a point with fixed coordinates, or by finding reference mark which may be a spot of dark dye. In the case where a reference mark is employed, a dye with a desired contrast is deposited in a specific position on the disc with respect to two clusters of cells. The optical disc reader is then directed to skip to the center of one of the clusters of cells and the standard rectangle is then centered around the selected cluster.

As for the user options mentioned above in regard to step 246, the user may specify a desired sample area shape for cell counting, such as a rectangular area, by direct interaction with mouse selection or otherwise. In the present embodiment of the software, this involves using the mouse to click and drag a rectangle over the desired portion of the optical bio-disc-derived image that is displayed on a monitor 114. Regardless of the evaluation area selection method, a respective rectangular area is evaluated for counting in the next step 248.

The third principal step in FIG. 21 is step 248, which is directed to background illumination uniformization. This process corrects possible background uniformity fluctuations caused in some hardware configurations. Background illumination uniformization offsets the intensity level of each sample point such that the overall background, or the portion of the image that is not cells, approaches a plane with an arbitrary background value V_{background}. While V_{background} may be decided in many ways, such as taking the average value over the standard rectangular sample area, in the present embodiment, the value is set to 2000. The value V at each point P of the selected rectangular sample area is replaced with the number (V_{background}+(V−average value over the neighborhood of P)) and truncated, if necessary, to fit the actual possible range of values, which is 0 to 4095 in a preferred embodiment of the present invention. The dimensions of the neighborhood are chosen to be sufficiently larger than the size of a cell and sufficiently smaller than the size of the standard rectangle.

The next step in the flow chart of FIG. 21 is a normalization step 250. In conducting normalization step 250, a linear transform is performed with the data in the standard rectangular sample area so that the average becomes 2000 with a standard deviation of 600. If necessary, the values are truncated to fit the range 0 to 4096. This step 250, as well as the background illumination uniformization step 248, makes the software less sensitive to hardware modifications and tuning. By way of example and not limitation, the signal gain in the detection circuitry, such as top detector 158 (FIG. 18), may change without significantly affecting the resultant cell counts.

As shown in FIG. 21, a filtering step 252 is next performed. For each point P in the standard rectangle, the number of points in the neighborhood of P, with dimensions smaller than indicated in step 248, with values sufficiently distinct from V_{background} is calculated. The points calculated should approximate the size of a cell in the image. If this number is large enough, the value at P remains as it was; otherwise it is assigned to V_{background}. This filtering operation is performed to remove noise, and in the optimal case only cells remain in the image while the background is uniformly equal V_{background}.

An optional step 254 directed to removing bad components may be performed as indicated in FIG. 21. Defects such as scratches, bubbles, dirt, and other similar irregularities may pass through filtering step 252. These defects may cause cell counting errors either directly or by affecting the overall distribution in the images histogram. Typically, these defects are sufficiently larger in size than cells and can be removed in step 254 as follows. First a binary image with the same dimensions as the selected region is formed. A in the binary image is defined as white, if the value at the corresponding point of the original image is equal to V_{background}, and black otherwise. Next, connected components of black points are extracted. Then subsequent erosion and expansion are applied to regularize the view of components. And finally, components that are larger than a defined threshold are removed. In one embodiment of this optional step, the component is removed from the original image by assigning the corresponding sample points in the original image with the value V_{background}. The threshold that determines which components constitute countable objects and which are to be removed is a user-defined value. This threshold may also vary depending on the investigational feature being counted i.e. white blood cells, red blood cells, or other biological matter. After optional step 254, steps 248, 250, and 252 are preferably repeated.

The next principal processing step shown in FIG. 21 is step 256, which is directed to counting cells by bright centers. The counting step 256 consists of several sub steps. The first of these sub steps includes performing a convolution. In this convolution sub step, an auxiliary array referred to as a convolved picture is formed. The value of the convolved picture at point P is the result of integration of a picture after filtering in the circular neighborhood of P. More precisely, for one specific embodiment, the function that is integrated, is the function that equals v-2000 when v is greater than 2000 and 0 when v is less than or equal to 2000. The next sub step performed in counting step 256 is finding the local maxima of the convolved picture in the neighborhood of a radius about the size of a cell. Next, duplicate local maxima with the same value in a closed neighborhood of each other are avoided. In the last sub step in counting step 256, the remaining local maxima are declared to mark cells.

In some hardware configurations, some cells may appear without bright centers. In these instances, only a dark rim is visible and the following two optional steps 258 and 260 are useful.

Step 258 is directed to removing found cells from the picture. In step 258, the circular region around the center of each found cell is filled with the value 2000 so that the cells with both bright centers and dark rims would not be found twice.

Step 260 is directed to counting additional cells by dark rims. Two transforms are made with the image after step 258. In the first substep of this routine, sub step (a), the value v at each point is replaced with (2000-v) and if the result is negative it is replaced with zero. In sub step (b), the resulting picture is then convolved with a ring of inner radius R1 and outer radius R2. R1 and R2 are, respectively, the minimal and the maximal expected radius of a cell, the ring being shifted, subsequently, in sub step (d) to the left, right, up and down. In sub step (c), the results of four shifts are summed. After this transform, the image of a dark rim cell looks like a four petal flower. Finally in sub step (d), maxima of the function obtained in sub step (c) are found in a manner to that employed in counting step 256. They are declared to mark cells omitted in step 256.

After counting step 256, or after counting step 260 when optionally employed, the last principal step illustrated in FIG. 21 is a results output step 262. The number of cells found in the standard rectangle is displayed on the monitor 114 shown in FIGS. 1 and 5, and each cell identified is marked with a cross on the displayed optical bio-disc-derived image.

Additional computer science methodologies and apparatus directed to extracting and visualizing data from bio-discs and/or optical analysis discs are discussed in commonly assigned U.S. patent application Ser. No. ______ entitled “Method and Apparatus for Visualizing Data” filed Jan. 13, 2003 and U.S. patent application Ser. No. ______ entitled “Methods and Apparatus for Extracting Data From an Optical Analysis Disc” filed on Jan. 14, 2003 both of which have been herein incorporated by reference.

Synthetic Nanoparticles and Nanocapsules

A particle of a few micrometers in size is detectable with a CD and DVD disc drive. If a particle is used as a reporter in a bio-disc assay there are density and size requirements specific for an assay such that the particles in buffer of pre-determined density move with proper speed at a given centrifugal force through the channel of the disc in the drive. If the particles are too light, they move too slow and full separation of non-bound and bound particles does not occur. If the particles are too heavy, they move too fast and may have too much mass such that their movement can result in breaking of specifically bound microparticles or nanoparticles.

There are two major technical challenges when synthesizing particles as labels for a disc assay. The first is how to get particles with narrow size and shape distribution. The second is how to incorporate bio-active substances such as enzymes and antibodies within the particles to thereby form a bio-particle or bio-active particle. It is an object of the present invention to synthesize bio-particles of essentially uniform size and shape.

A bio-active nanoparticle or bioparticle may be synthesized by polymerization from monomers to form a linear or cross-linked polymer particle. Polymerization is catalized by an initiator including, for example, a radical formed as a result of a decay of the initiator molecule under UV light or temperature action. Polymerization may occur fast as a chain reaction. As a result, particles of very big sizes and even continuous gel-like structures can form if the reaction is not stopped after some time. Even when the polymerization reaction is discontinued after a specific time, resulting particles may be of different shapes and sizes.

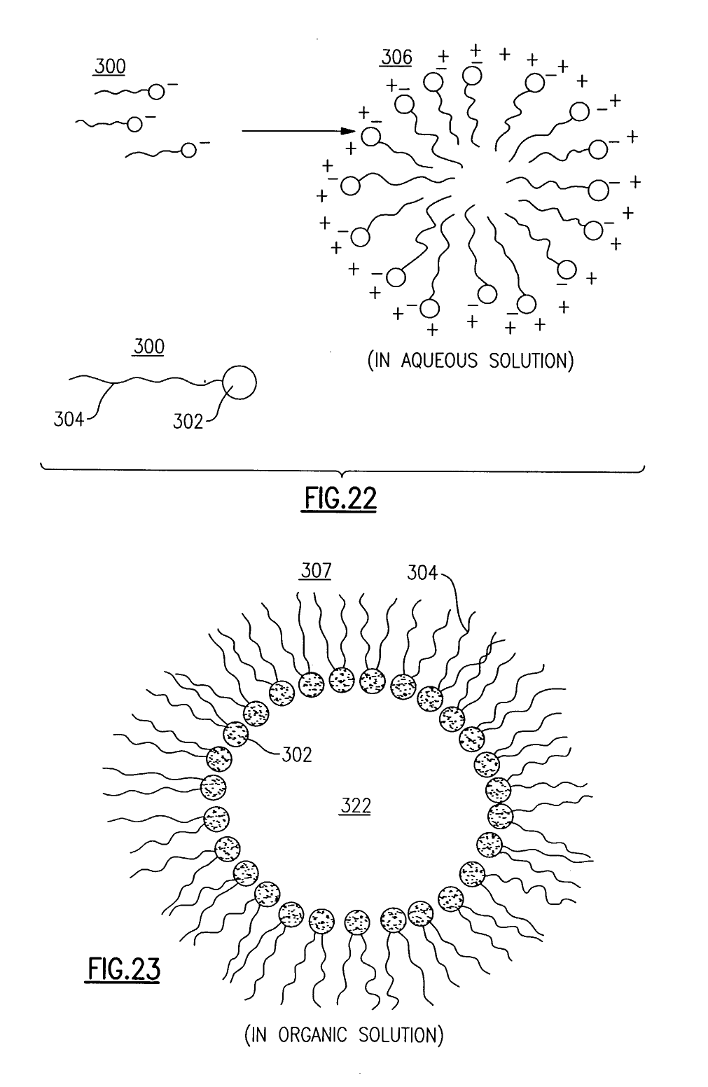

Synthesis of nanoparticles of essentially uniform size and shape may be achieved if polymerization is performed in the presence of a surfactant, emulsifier, or micelle-forming surfactant that forms small oil bubbles or micelles. Many surfactants form ‘micelles’ or ‘normal micelles’ in an aqueous solution. As shown in FIG. 22, in normal micelles, the surfactant 300 aggregates with its hydrophobic tail 304 forming the inside of the micelle 306 and its polar head 302 forms the outer polar shell.

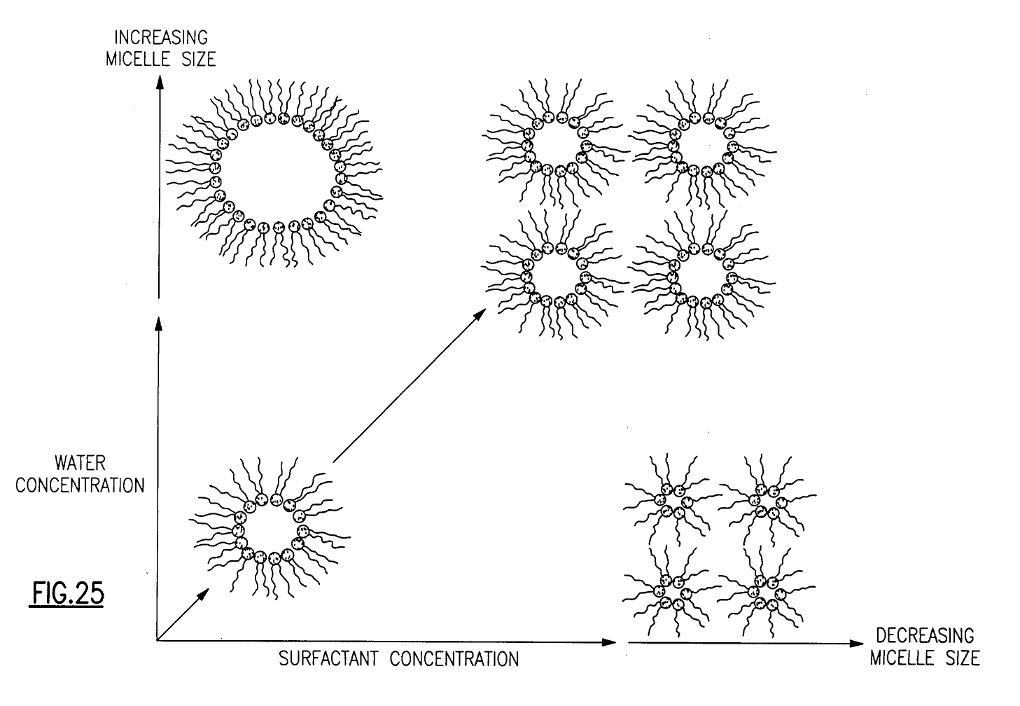

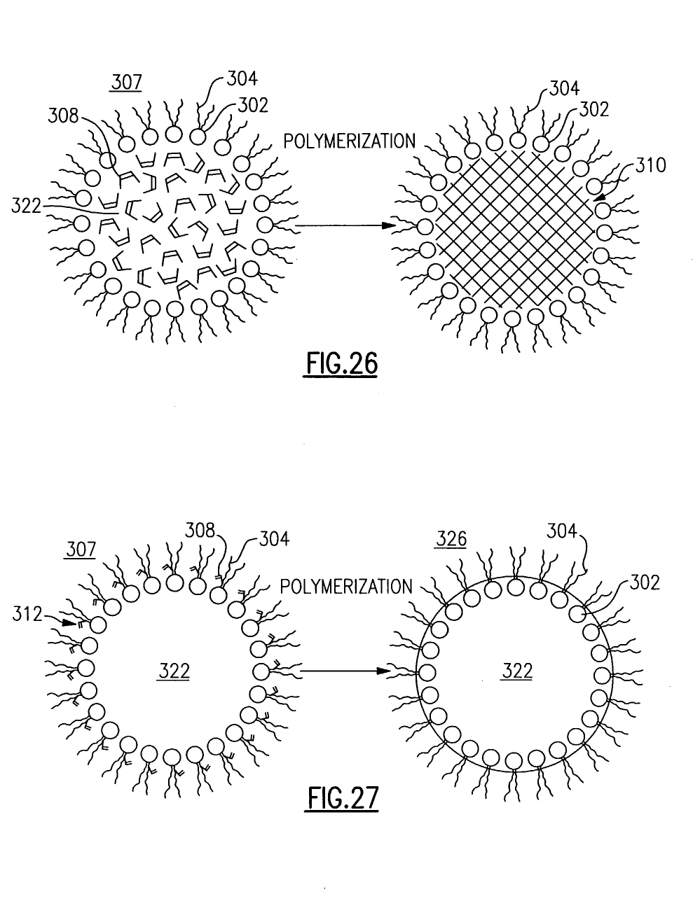

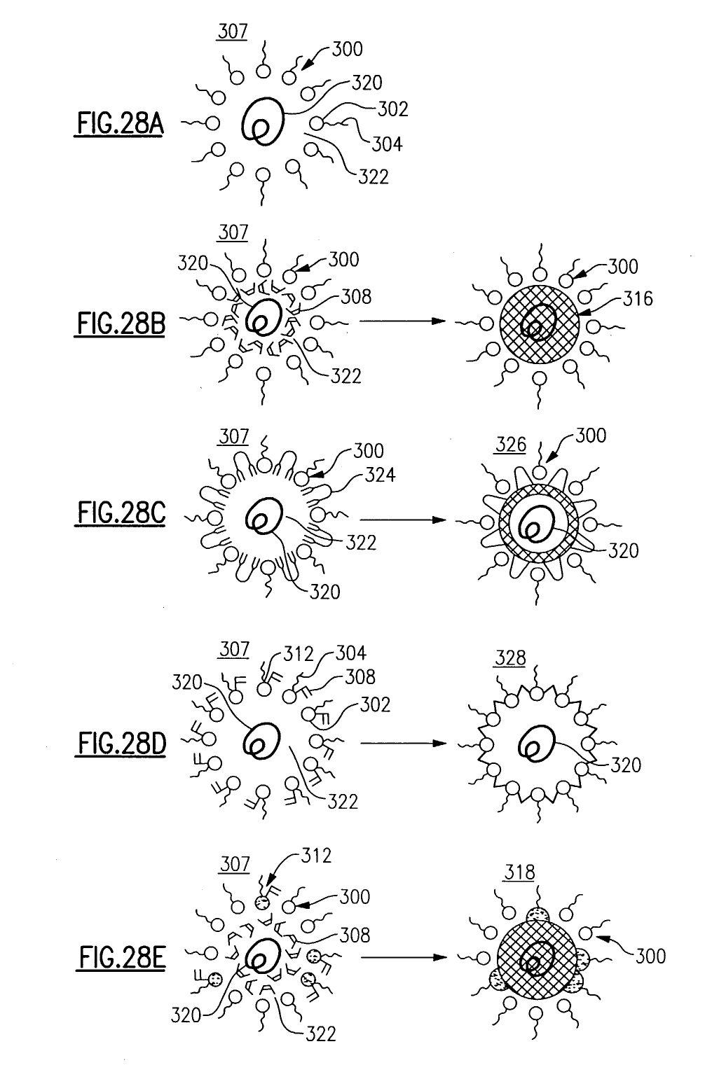

With reference next to FIG. 23 it is shown that some surfactants form reverse micelles (RMs) 307 in which the hydrophobic tail 304 forms the outer shell while the polar heads 302 of surfactant 300 forms a polar nucleus delimiting an aqueous inner cavity 322 in an organic solution. The organic solution may be a non-polar organic solvent including non-polar aliphatic, cycloaliphatic, or aromatic solvents; halogenated solvents such as chloroform; and hydrocarbons such as octane, iso-octane, hexane, toluene, or cyclohexane, or mixtures thereof. In one preferred embodiment of the present invention, nanoparticles are polymerized, formed, or synthesized inside RMs. Further details relating to the synthesis of nanoparticle inside RMs are discussed below in conjunction with FIG. 26 and in Example 1.

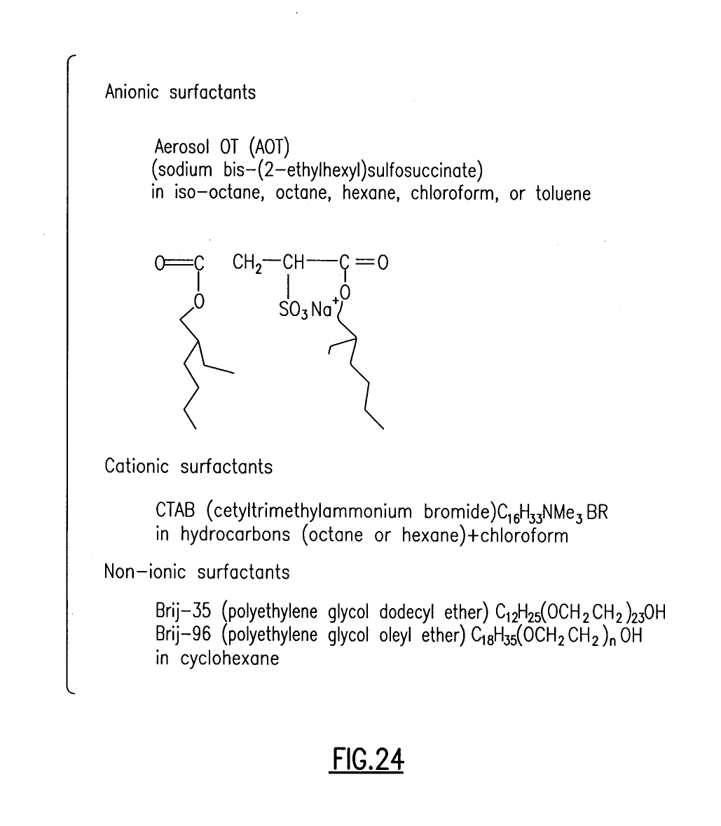

Surfactants that may be used to form reverse micelles include anionic, cationic, and non-ionic surfactants. The most common reverse micelle forming surfactant is an anionic surfactant bis-(2-ethylhexyl) sulfosuccinate sodium salt, (AOT). The structure of AOT is shown in FIG. 24. Other reverse micelle forming surfactants include, for example, cetyltrimethylammonium bromide (CTAB), polyethylene glycol dodecyl ether (Brij 35), polyethylene glycol oleyl ether (Brij 96), or any mixture thereof.

Surfactants may either form a microemulsion in non-polar organic solvents. Terminology of ‘micelles’ and ‘microemulsions’ is used according to the size of surfactant aggregates. Micelles normally range in size from approximately 1 to about 1000 nanometers, are stable, and result in a transparent or clear solution. Microemulsions, however, have aggregates that are about 1 micrometer or more in size and normally result in a cloudy solution and the aggregates are not so stable.

The diameter of the inner cavity 322 of reverse micelles 307 normally ranges from 1 nm to 1000 nm and can be varied by changing the degree of hydration (Wo) of the aqueous inner cavity 322. One of the important characteristics of the reverse micellar system is the degree of hydration (Wo) which is the ratio of water concentration and surfactant concentration, as expressed by the following formula:
Wo=[H₂O]/[surfactant]

This parameter defines the size of the reverse micelles. As illustrated in FIG. 25, the higher the Wo, the larger the micelles, and the larger the inner cavity 322 diameter. Reverse micelles solubilize various polar or water-soluble substances 320 including aqueous solutions of labels, dyes, drugs, and bio-active substances such as enzymes, antibodies, DNA, RNA, and proteins into the aqueous inner cavity 322 of RMs as illustrated and described, below, in conjunction with FIGS. 28A-28E. Reverse micelles also solubilize polymerizable mixtures of monomers 308, co-monomers, and cross-linking agents that under the effect of a polymerization initiator produce the polymeric nanoparticle 310 of the present invention as illustrated in FIG. 26. A preferred polymerizing mixture may include, for example, acrylic or methacrylic monomer compounds forming a linear or cross-linked polymer. The polymerizing mixture may optionally include cross-linking agents such as acrylamide (AA) and N,N′-methylene-bis-acrylamide (MBAA) or a mixture thereof. Polymerization initiators that may be used in the polymerization process include, for example, radicals formed as a result of a decay of the initiator molecule under UV light or temperature action.

The size of the synthesized polymeric nanoparticles 310 can be varied by changing the concentrations of the monomers 308, co-monomers, and initiator in the reverse micellar solution. Alternatively, the size of the inner cavity 322 of the reverse micelle 307 may be varied by changing its degree of hydration thus incorporating more polymerizable components including monomers 308, co-monomers, and initiator thereby increasing the size of the nanoparticle 310 formed after polymerization.

The size of polymeric nanoparticles 310 may increase after polymerization relative to the size of micelles before polymerization. This can be explained by the fact that besides intramicellar polymerization, intermicellar polymerization takes place to a certain extent. Despite the increase in size during polymerization, nanoparticles comprising polyacrylamide are still realtively small. An example of such particles are those obtained by polymerization of the mixture of acrylamide (AA) and N,N′-methylene-bis-acrylamide (MBAA) induced by UV-irradiation in the system of AOT micelles in toluene. The size of resulting particles, in this example, can be varied according to the polymerization conditions, from a few nanometers up to about 0.6 micrometers.

The micellar solution may became either more opaque or a polymer precipitate may appear after polymerization. The size of polymeric nanoparticles 310 is strongly influenced by the composition of the RM system as discussed above, but the formation of uniform species in each case makes it possible to easily select systems with particles of an appropriate size.

The polymeric nanoparticles 310 of the present invention may be modified for use in an optical-bio disc system. For instance, bigger particles that are detectable using an optical disc reader 112, FIG. 1, can be obtained using the polymerization technique described above. For example, particles having a diameter of 1 um can be detected using a CD-type optical disc reader such us a CD-R drive.

Another way to improve the signal is to change the particle material, or to add an absorbing or luminescing label to the micelle before polymerization, so that the label becomes embedded in the particle after polymerization. The label may also be covalently bound to the polymers of the nanoparticle 310. Dyes or labels can be easily incorporated into polymeric nanoparticles 310 when added to reverse micelles 307 before polymerization. Many dyes absorbing or fluorescing at a pre-determined wavelength are suitable for such purpose. In one preferred embodiment of the present invention, infrared absorbing dyes are embedded into nanoparticles 310. These infrared dyes absorb electromagnetic radiation at or near the wavelength of the laser of a CD player. In this case, the signal generated by the nanoparticles will be good despite small particle size.

Semiconductor nanoparticies may also be synthesized using the same methods described above. In this embodiment of the present invention, semiconductor components are mixed with the polymerizing solution in the RMs prior to polymerization thereby generating nanoparticles having semiconductor properties.

Another embodiment of the present invention is the synthesis of magnetic nanoparticles using the same RM methods described above. In this embodiment metallic substances such as iron oxide are mixed with the polymerizing solution in the RMs prior to polymerization thereby generating magnetic nanoparticles. Further details relating the use of magnetic particles in bio-disc assays are described in, for example, commonly assigned and co-pending U.S. patent application Ser. No. 10/307,263 entitled “Magneto-Optical Bio-Discs and Systems Including Related Methods” filed on Nov. 27, 2002 which is herein incorporated by reference in its entirety.

Yet another embodiment of the present invention is the synthesis of nanocapsules 326, illustrated in FIG. 27, from micelle-forming polymer conjugated surfactants or polymerizable surfactants 312 having attached thereto monomer 308. Monomer 308 cross-link with each other during polymerization thereby forming nanocapsules with an aqueous inner cavity 322. The inner cavity 322 may be used as a compartment for water-soluble bio-active substances 320 that may be used in bio-disc assays of the present invention as discussed below in conjunction with FIGS. 28A-28E.

Any nanoparticle 310 or nanocapsule 326 synthesized using the methods of the present invention can be coated with a chemically active substance for conjugation to bio-active molecules or agent and used in disc assays. Suitable bio-compatible molecules include, for instance saccharide material such as dextran, cellulose, or protein material. Further details relating methods for conjugating bio-active molecules onto various surfaces are described in, for example, commonly assigned and co-pending U.S. patent application Ser. No. ______ entitled “Multi-Purpose Optical Analysis Disc for Conducting Assays and Related Methods for Attaching Capture Agents” filed on Jan. 21, 2003 which is herein incorporated by reference in its entirety. In addition, synthesis of a nanoparticle 310 or nanocapsule 326 having free active groups can be achieved by using monomers with such groups for polymerization.

Alternatively, the polymeric nanoparticle may be modified by grafting active groups onto the nanoparticle surface. See for instance Braybrook et al., Prog. Polym. Sci. 15:715-734, 1990. Most of the modification procedures known in the art involve sequential treatment of surfaces with chemical reagents. Examples include sulfonation of polystyrene, Gibson, et al., Macromolecules 13:34, 1980; base hydrolysis of polyimide, Lee, et al., Macromolecules 23:2097, 1990; and base treatment of polyvinylidene fluoride, Dias et al., Macromolecules 17:2529, 1984. Another conventional method for modifying polymer surfaces includes exposing the surface of the hydrocarbon such as polyethylene with nitrene or carbene intermediates generated in a gas phase (Breslow in “Azides and Nitrenes”, Chapter 10, Academic Press, New York, 1984). Perfluorophenyl azides (PFPAs) have been shown to be efficient in the insertion in CH bonds over their non-fluorinated analogues (Keana, et al., Fluorine Chem. 43:151,1989). Recently, bis-(PFPA)s have been shown to be efficient cross-linking agents for Polystyrene (Cai, et al., Chem. Mater. 2:631,1990).

Chemical modification of the inert polymer substrate surface, such as the surface of a nanoparticle 310, is efficiently done through grafting procedures that allow the deposition of a thin interphase layer, active layer, or interlayer on the surface of the nanoparticle 310. Ideally, the interphase layer should make a stable linkage of the grafted material to the substrate surface and contain a spacer molecule ending in a functional group or variety of chemically different functional groups. This allows the selection of specific surface chemistries for efficient covalent immobilization of a variety of bio-active agents with different demand for spatial orientation, side directed attachment within the structure of the binding protein. The introduction of spacer molecules, especially hydrophilic spacers as part of the graft, contributes significantly to the flexibility and accessibility of the immobilized bio-active substance or agents. By placing a spacer layer between the solid phase of the nanoparticle surface modified or grafted with different functional groups and the bio-active substance or agent, a potentially denaturing effect of the direct contact of the bio-active agent with the functional groups is eliminated.

Integration of Bio-Active Substances into Nanoparticles and Nanocapsules

With reference now generally to FIGS. 28A to 28E, the present invention also relates to bio-active nanoparticles 316 and 318 which are complexes having biologically active or bio-active substances immobilized within the nanoparticles 310 as described below in conjunction with FIGS. 28B and 28E. Alternatively, the bio-active substances may be encapsulated inside nanocapsules (bio-active nanocapsules 326 and /328) as described below in connection with FIGS. 28C and 28D. In another alternative, the bio-active substances may be immobilized on the surface of the nanoparticles 310. The resulting bio-active complexes are preferably capable of producing a detectable signal or of being detected by suitable means. For this reason, they are useful as reagents for bio-disc assays and disc analytical methods. The bio-active substances may include, but are not limited to, proteins, antigens, antibodies, enzymes, drugs, DNA, RNA, functionally active subunits, parts and mixtures thereof.

Immobilized biologically active substances, antibodies and enzymes, in particular, are widely used in biotechnology. A great number of carriers with different properties have been developed for immobilization of bio-active substances. However, most of the carriers used suffer from the fact that their properties, particularly large size, cannot be varied to a great extent. A problem of diminishing the size of carrier particle stems from the necessity of increasing the surface of a catalyst and overcoming diffusion limitations. The use of small colloid particles would allow combining the advantages of heterogeneous and homogeneous catalysis. In a preferred embodiment of the present invention, a single bio-active substance is immobilized inside its own nanoparticle 310 or contained in a single nanocapsule 326.

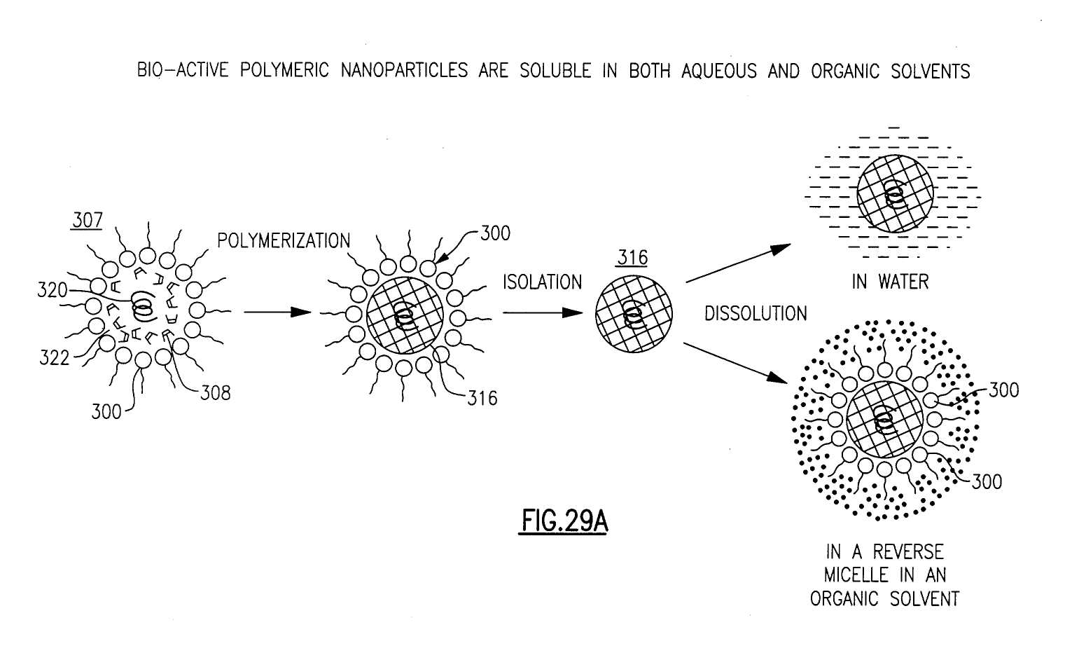

Bio-active substances can be immobilized into the nanoparticles 310 if they are solubilized inside the aqueous inner cavity 322 of the reverse micelle 307 and polymerization is performed in their presence. Biological properties of bio-active substances such as proteins and drugs entrapped in nanoparticles 310 may change, but is it known that bioactivity (for example, enzyme catalytic activity) can be retained after polymerization up to 90% of initial activity. One advantage in using the nanoparticles of the present invention for bio-disc assays is the substantial improvement in the stability of a bio-active substance due to protection by the polymeric layer of the nanoparticle 310 from denaturing effects of temperature and aggressive solvents. For example, nanoparticle embedded enzymes possess high thermostability (exceeding by a factor of 1000 the thermostability of the native enzyme) and are soluble and stable both in aqueous solutions and non-polar organic solvents as described below in connection with FIGS. 29A and 29B. Bio-active substances that are sensitive to denaturation may alternatively be encapsulated in a nanocapsule to retain its biological activity while protecting it from denturation from solvents. Synthesis of nanocapsules is described below in conjunction with FIGS. 28C and 28D.

Referring now specifically to FIG. 28A, there is shown a graphical representation of a reverse micelle 307 containing a water-soluble substance 320 entrapped in the inner aqueous layer 322. The water-soluble substance shown in FIGS. 28A-28E may be a label or dye, magnetic particles or colloids, semiconductor materials, or bio-active substances.

Referring next to FIG. 28B, there is illustrated a schematic representation of the formation of a modified nanoparticle 316. The modified nanoparticle 316 is a nanoparticle 310 containing an immobilized water-soluble substance 320. The first step in the formation or synthesis of the modified nanoparticle 316 is the solubilization of monomers 308 and a water-soluble substance 320 into the inner cavity 322 of the RM 307. Polymerization of the monomers 308 is then carried out using an initiator. The initiator is preferably an UV activated initiator. The water-soluble substance is incorporated in the matrix of the nanoparticle polymer 316 during the polymerization process. If the water-soluble substance 320 in the modified nanoparticle 316 is a bio-active substance, then the nanoparticle is herein referred to as a “bio-active nanoparticle” since it has biological activity.

With reference to FIG. 28C, there is shown a schematic representation of the formation of a nanocapsule 326 containing a water-soluble substance 320. The nanocapsule 326 is synthesized from weakly polar monomers 324 solubilized near the shell of a RM 307. The weakly polar monomers 324 forms a shell around the inner cavity 322 of the RM 307 upon polymerization due to their location thereby entrapping the water-soluble substance 320. If the water-soluble substance 320 in the nanocapsule 326 is a bio-active substance, then the nanocapsule is herein referred to as a “bio-active nanocapsule” since it has biological activity.

In another embodiment of the present invention, a water-soluble substance 320 may be encapsulated in a hydrophobized nanocapsule 328. FIG. 28D is a schematic representation of the formation of the hydrophobized nanocapsule 328 containing the water-soluble substance 320. The hydrophobized nanocapsule may be synthesized from a micelle-forming polymerizable surfactant 312. Polymerizable surfactants are surfactants having a monomer 308 attached thereto such that when polymerization is carried out, the polymerizable surfactants 312 are linked together forming the hydrophobized nanocapsule 328. Any water-soluble substance 320 entrapped in the inner cavity 322′ of the RM 307 are encapsulated inside the hydrophobized nanocapsule 328 after polymerization as illustrated. If the water-soluble substance 320 in the hydrophobized nanocapsule 328 is a bio-active substance, then the nanocapsule is herein referred to as a “bio-active hydrophobized nanocapsule” since it has biological activity.

Referring now to FIG. 28E, there is shown a schematic representation of the formation of a surface-modified or hydrophobized nanoparticle 318 containing an immobilized substance 320. The hydrophobized nanoparticle is formed from co-polymerization of micelle-forming polymerizable or monomeric surfactants 312 and water-soluble monomers 308. The hydrophobicity of the synthesized hydrophobized nanoparticle 318 is dependent upon the concentration of the polymerizable surfactants 312 present during polymerization. As discussed above in FIG. 28B, if the water-soluble substance 320 in the hydrophobized nanoparticle 318 is a bio-active substance, then the nanoparticle is herein referred to as a “bio-active hydrophobized nanoparticle” since it has biological activity.

With reference next to FIG. 29A, there is illustrated the polymerization or synthesis, isolation, and dissolution of a bio-active nanoparticle 316 having an enzyme 320 embedded therein. The bio-active nanoparticle 316 is synthesized as described above in connection with FIG. 28B. Once the bio-active nanoparticle 316 is formed (after polymerization), the bio-active nanoparticle 316 is isolated by salting out or precipitation. Precipitation is carried out by adding an excess of cold acetone to the micellar solution containing the newly synthesized nanoparticles. The amount of excess acetone is preferably 10 fold. The resulting solution containing excess cold acetone is centrifuged and the supernatant is discarded. The pellet containing the nanoparticles is then washed at least once with cold acetone, the supernatant discarded and, the pellet dried for storage. Once dried, the particles exist in a powdered form. The temperature of the cold acetone is preferably less than 4 degrees C. Cold acetone is used to prevent denaturation of the enzyme 320 during isolation of the nanoparticle 316. Once the bio-active nanoparticle 316 is dried, however, the enzyme 320 normally remains stable for an extended period of time. The powdered nanoparticles may then be dissolved directly into an aqueous solution or into an organic solvent with the appropriate concentration of surfactants for use in bio-disc assays.

FIG. 29B is an illustration of the synthesis or polymerization, isolation, and dissolution of a bio-active hydrophobized nanoparticle 318 having an enzyme 320 embedded therein. The bio-active nanoparticle 318 is synthesized as described above in connection with FIG. 28E. The isolation of the resulting bio-active hydrophobized nanoparticle 318 is similar to the steps described above in conjunction with FIG. 29A. Since the resulting bio-active hydrophobized nanoparticle 318 is slightly hydrophobic due to attached polymerizable surfactant 312 on its surface, surfactants 300 may be needed to dissolve a desired amount of nanoparticles in an aqueous solution. The hydrophobized bio-active nanoparticle 318 is encapsulated in a micelle double layer 330 in an aqueous solution due to the orientation of the attached polymerizable surfactant 312. Similarly, surfactants are also needed to solubilize the hydrophobized bio-active nanoparticle 318 in an organic solvent since the hydrophobized bio-active nanoparticle 318 is slightly polar.

Immunochemical Assays Using Bio-Active Nanoparticles on the Optical Bio-Disc

There are three general classes of binding assays as related to the present invention. These include binding protein capture assays, analyte capture assays, and sandwich type assays. The latter assay type can have a binding protein-analyte-binding protein or analyte-binding protein-analyte format.

A specific implementation of a binding assay is an immunoassay. In such an immunoassay, the binding protein may be represented by a capture antibody or a capture antigen and the analyte may be an antigen/hapten or a target antibody, respectively. The product of the reaction is an antigen-antibody immune complex.

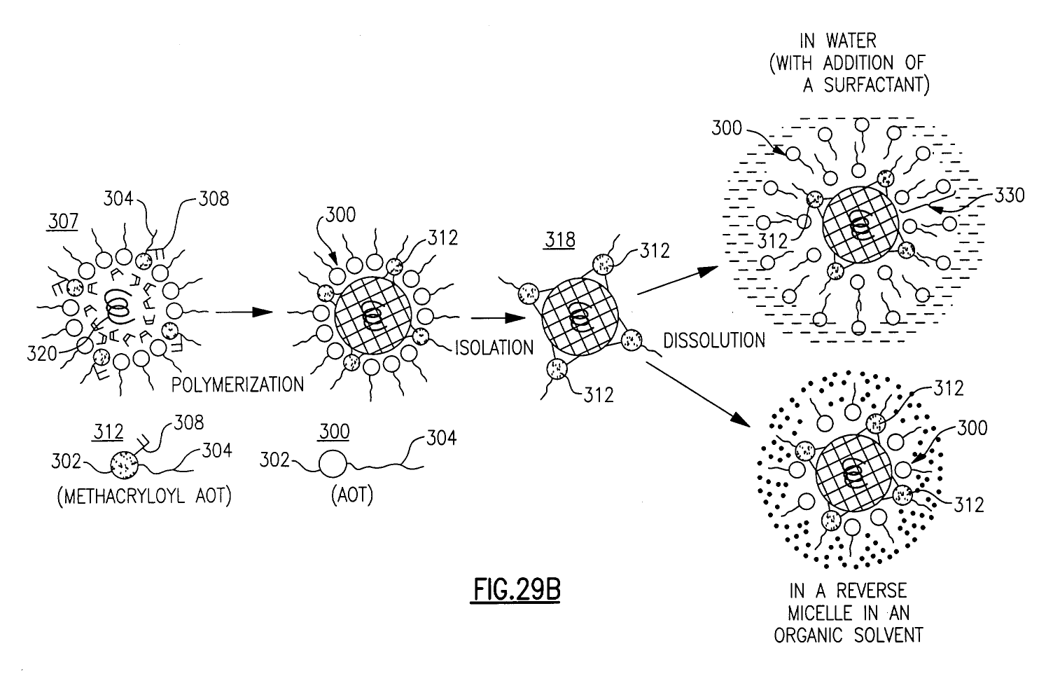

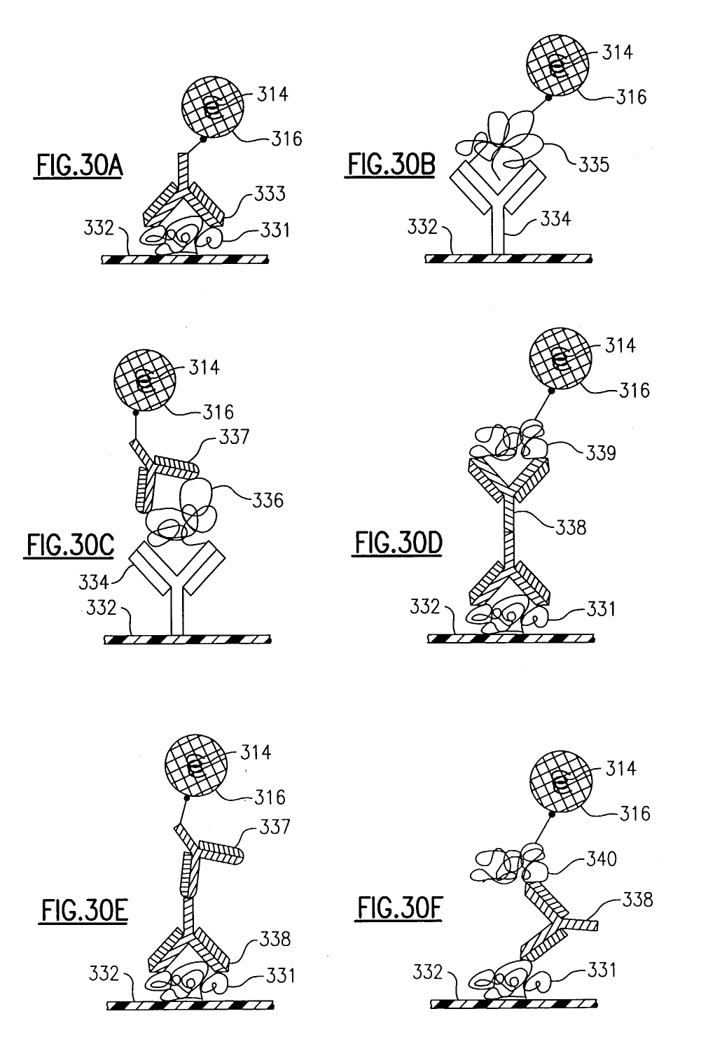

The following discussion will concentrate on the immunoassay implementation of binding assays but will in most cases apply also to the broader definition of binding assays. More detailed information on immunoassays can be found in “Radioimmunoassay Methods”, K. E. Kirkham and W. M. Hunter (Eds.), Churchill Livingston Edinburgh and London (1973) and “Principles of Competitive Protein Binding Assays”, W. D., Odel, W. H. Daughaday, JB Lippincot Co., Philadephia, Pa. (1971) which is herein incorporated by reference in its entirety. Both, a target or analyte antigen and a target antibody can be quantified by an immunoassay designed in analogy to one of the formats as described below in conjunction with FIGS. 30A-30F. The antigens and antibodies, as illustrated and described in conjunction with FIGS. 30A-30F, are numbered according to their functional characteristics.

Referring now specifically to FIG. 30A, there is illustrated an antibody capture assay utilizing a capture antigen 331 attached to a solid support 332. A tagged analyte antibody 333 and its untagged analog are allowed to competitively bind to the immobilized capture antigen 331. The analyte antibody 333 is tagged or conjugated with a bio-active nanoparticle 316. Bio-active nanoparticle 316 contains an enzyme that reacts with a substrate to produce a detectable signal. The concentration of analyte antibody is determined by the comparison of signal obtained with known standards of the analyte antibody.

Referring next to FIG. 30B, there is shown a pictorial representation of an antigen capture assay. In this embodiment of the present invention, capture antibody 334 is immobilized on the solid support 332 and a tagged analyte antigen 335 and its untagged analog are allowed to competetively bind to the capture antibody 334 on the solid support 332. The analyte antigen 335 is tagged or conjugated with a bio-active nanoparticle 316. Bio-active nanoparticle 316 contains an enzyme that reacts with a substrate to produce a detectable signal. The concentration of analyte antigen is determined by the comparison of signal obtained with known standards of the analyte antigen.

With reference now to FIG. 30C, there is depicted an antibody-analyte-antibody sandwich assay wherein a capture antibody 334 is bound to the solid support 332 and analyte antigen 336 is allowed to bind to the capture antibody 334. The amount of bound analyte 336 is then determined through binding and measurement of signal generated from the tagged signal antibody 337. The signal antibody 337 is tagged or conjugated to a bio-active nanoparticle 316. Bio-active nanoparticle 316 contains an enzyme that reacts with a substrate to produce a detectable signal. The concentration of analyte antigen 336 is determined by the comparison of signal obtained with known standards of the analyte antigen.

Conversely, an antigen-antibody-antigen sandwich assay (FIG. 30D) has a solid phase 332 having the capture antigen 331, bound thereto, which captures analyte antibody 338. Subsequently a tagged form of signal antigen 339 binds to the available free antibody binding sites of the analyte antibody 338 completing the antigen-antibody-labeled antigen sandwich. Examples of this assay are illustrated in FIGS. 30D and 30F which are based on the multivalency of immunoglobulin D and bivalency of IgG or IgD, respectively.

Quantification of antigen molecules is preferably most efficiently done by the two-antibody sandwich assay represented by FIG. 30C. The capture antibody 334 is immobilized on the solid support 332 and the signal antibody 337 is tagged or conjugated to a bio-active nanoparticle 316. The recognition of the same antigen by two different binding antibodies, namely the solid phase capture antibody 334 and the reporter linked signal or enumerating antibody 337, contributes to the exquisite specificity of the assay. The capture antibody 334 identifies a first epitope on the surface of the analyte molecule 336 while signal antibody 337 recognizes a second epitope at a different location on the surface of the same analyte antigen or molecule 336. The signal generated by the capture antibody-antigen-signal antibody complex is proportional to the amount of the bridging analyte antigen 336 present in the sample. The concentration of antigen in the analyzed specimen can then be determined through comparison with the signal generated by known quantity of pure antigen. An example of an assay based on this technique using radioiodine I-125 labeled antibody for detection of the antigen associated with serum hepatitis is disclosed in, for example, U.S. Pat. No. 3,867,517 which is incorporated herein by reference in its entirety.

Detection or quantification of an antibody or any immunoglobulin is alternatively done by a solid phase immobilized antigen test device, as shown in FIG. 30E. An analyte or target antibody 338 is allowed to bind to the capture antigen 331 creating an immobilized antigen-antibody complex. A tagged form of an anti-immunoglobulin signal antibody 337 or other immunoglobulin specific binding protein such as protein A and protein G, is then applied to the immobilized antigen-antibody complex which enumerates the analyte antibody 338 through binding of the signal antibody 337 to a site other than the epitope binding site of the analyte antibody 338. Detection of the signal generated directly or indirectly by the tagged reporter or signal antibody 337 becomes a measure for the presence and quantity of the analyte antibody 338 when comparison with a known reference material for the immunoglobulin is established.

More recently, antibodies are determined by antigen sandwich, dubbed “inverse sandwich” immunoassays as illustrated in FIG. 30F. This assay makes use of the presence of two equal epitope binding sites on each immunoglobulin G (IgG) molecule, thus allowing for a simultaneous binding of the analyte antibody 338 to two separate antigens, solid phase bound capture antigen 331 and reporter antigen 340. Reporter 340 represents the tagged form of capture antigen 331. Lateral flow antigen sandwich immunoassays have one antigen/hapten immobilized to a solid phase, most frequently a nitrocellulose or nylon membrane, and the second antigen, carrying the same epitope as the solid phase bound antigen, labeled with a bio-active nanoparticle 316. The bio-active nanoparticle may contain an enzyme, a radioisotope, a dye, or other signal generating substance. Antibody specific to the epitope represented by both antigens can than be specifically detected in a single step assay procedure.

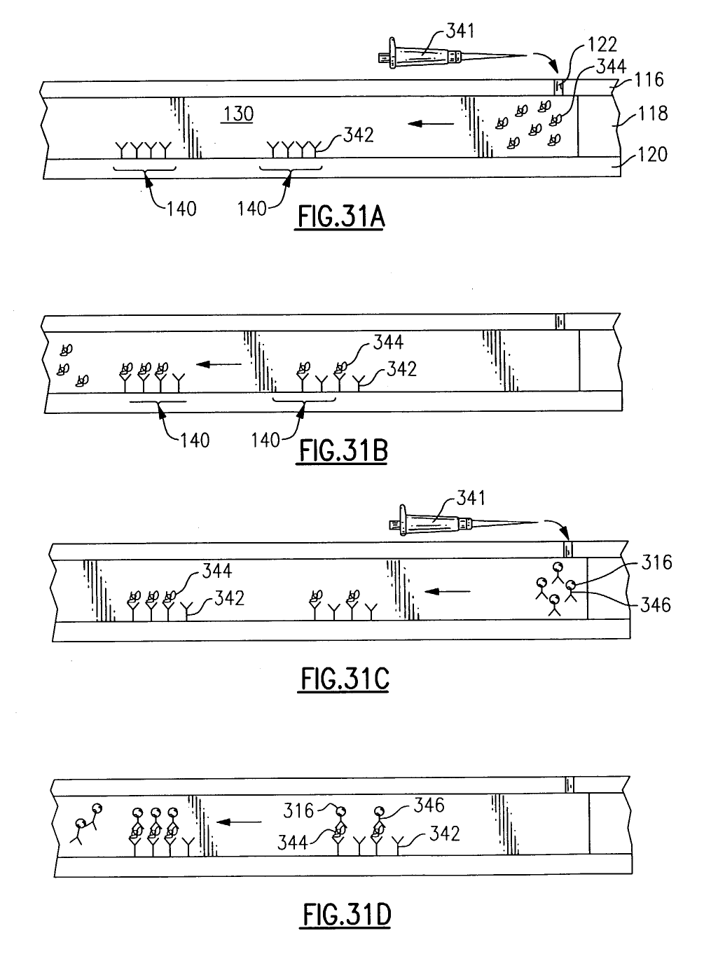

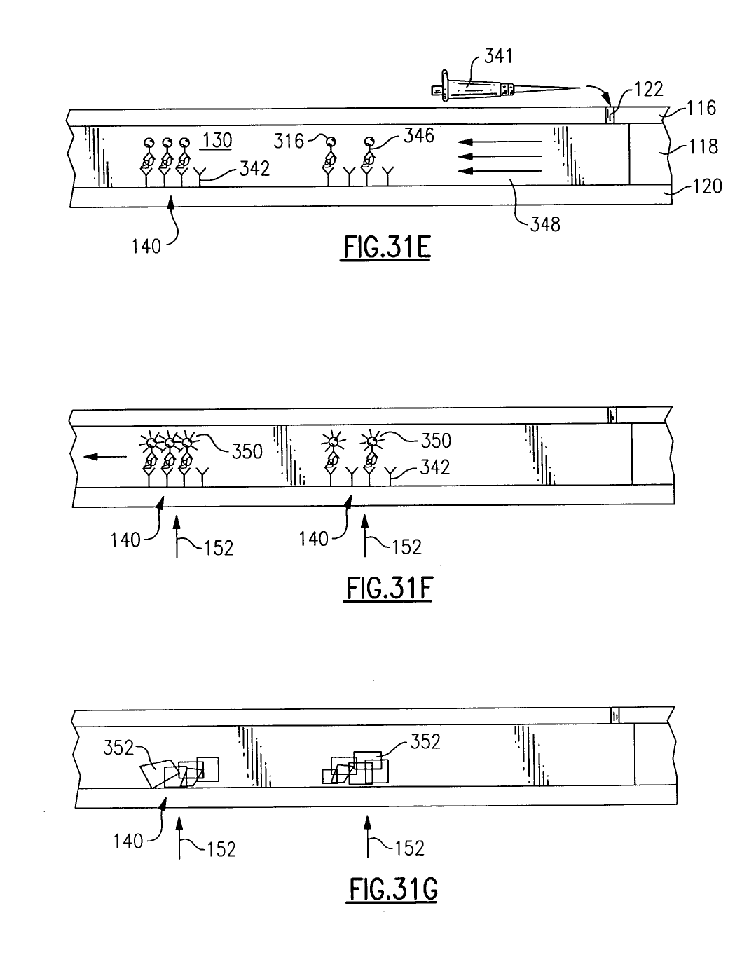

Turning now to FIGS. 31A to 31G, there is illustrated a method for detecting or determining the presence of target antigen or agent 344 in a sample using the bio-active nanoparticle 316 of the present invention in conjunction with the optical bio-disc 110. As shown in FIGS. 31A-31G and discussed above in conjunction with FIGS. 2, 5, and 10, the optical bio-disc 110 includes the cap portion 116, the adhesive member or channel layer 118, and the substrate 120. The disc format may be either the reflective disc format or the transmissive disc format with varying elements to each respective cap portion 116 and substrate 120 as described in conjunction with FIGS. 4, 9, 14, and 15. Although the disc composition between the different disc formats may vary, the biochemical interactions remain the same. The disc format for this invention may also include the e-rad format or the spiral analysis zone discs disclosed in, for example, the above incorporated commonly assigned and co-pending U.S. patent application Ser. No. 10/347,155 entitled “Optical Discs Including Equi-Radial and/or Spiral Analysis Zones and Related Disc Drive Systems and Methods” filed on Jan. 15, 2003.

Referring now specifically to FIG. 31A, a pipette 341 is loaded with a test sample with or without the target agent 344. The test sample is injected or deposited into the flow channel 130 through the inlet or injection port 122. As the flow channel 130 is further filled with test sample, the target agent 344 begins to flow or move down the flow channel 130 as illustrated in FIGS. 31A and 31B. If the analyte of interest is present in the test sample, the analyte or target agent 344 binds specifically to the capture agent 342 as shown in FIG. 31B. In this manner, the target agent 344 is retained within the target zone 140. Binding may be further facilitated by heating the disc or localized heating of the flow channel.

After binding, the flow channel 130 may be washed to clear the target zone 140 of any unattached target agents in the sample. After removing the unattached target agents in the sample, signal agents, probes, or antibodies 346 conjugated to the bio-active nanoparticle 316 are introduced in the flow channel 130, FIG. 31C. The bio-active nanoparticle 316 contains an enzyme 314 that reacts with a substrate to produce a detectable signal (FIG. 30C). As the flow channel 130 is filled with bio-active nanoparticles 316, the nanoparticles 316 begin to flow or move down the flow channel 130 as illustrated in FIGS. 31C and 31D. When the signal agents 346 comes into close proximity with the target 344, bound in the target zone 140 by the capture agent 342, the signal agents 346 bind specifically to the target agent 344 as illustrated in FIG. 31D, thereby immobilizing the bio-active nanoparticles 316 on the target zone 140.

After the signal agent binding step, the flow channel 130 may be washed to clear the target zone 140 of any unattached nanoparticles 316. Upon removal of unattached nanoparticles 316, enzyme-reactive substrates 348 are then introduced in the channel as shown in FIG. 31E. As the flow channel 130 is filled with enzyme substrate 348, the enzyme substrate 348 begin to flow or move down the flow channel 130 as illustrated in FIG. 31E. When the substrate comes in contact with the bio-active nanoparticle 316, containing enzyme 314, an enzyme-substrate reaction 350 occurs which results in the production of signal agents as shown in FIG. 31F. The signal agent may be color production, fluorescence, or luminophore production. The signal agent may also be a precipitate 352 as illustrated in FIG. 31G. The incident or interrogation beam 152 may then be scanned through the target zone 140 to determine the presence of signal agents as illustrated in FIGS. 31F and 31G. In the event no target agent 344 is present in the test sample, no enzyme substrate reaction 350 will occur and the signal agents will not be present. In this case, when the interrogation beam 152 is directed into the target zone 140, a zero or baseline reading will result thereby indicating that no target 344 was present in the sample.

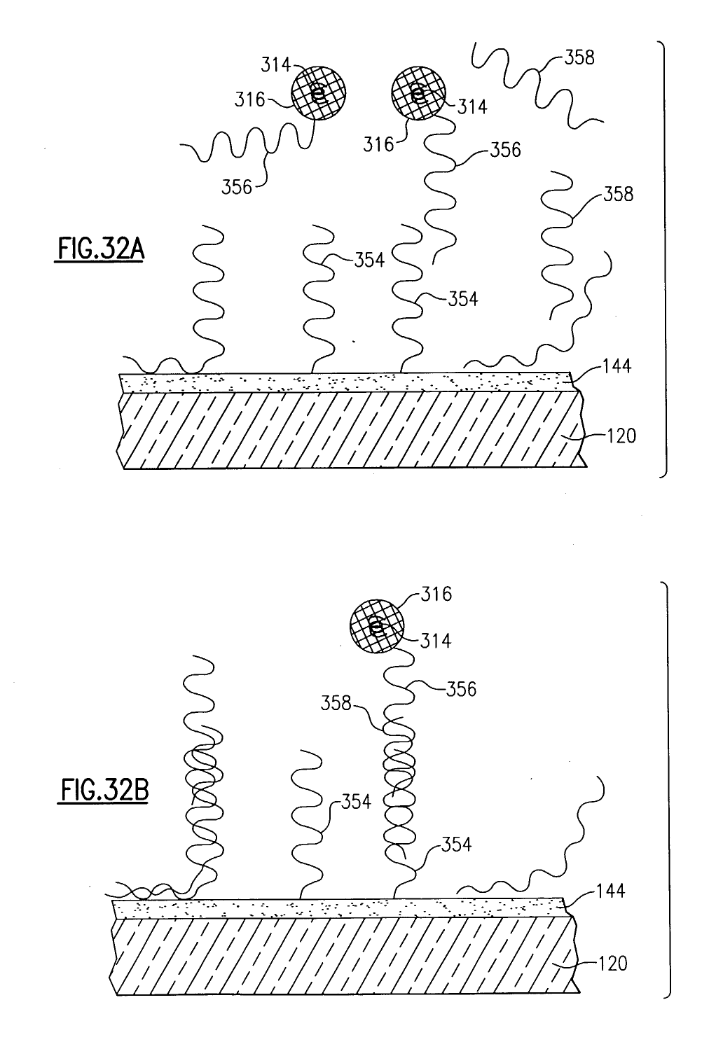

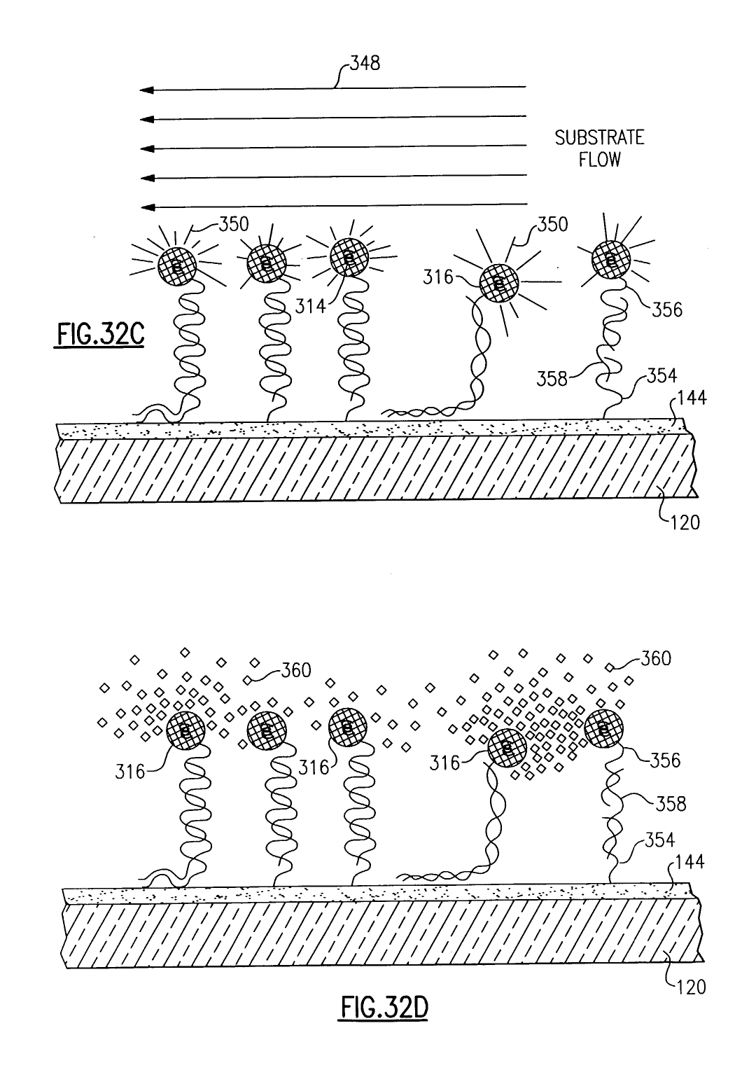

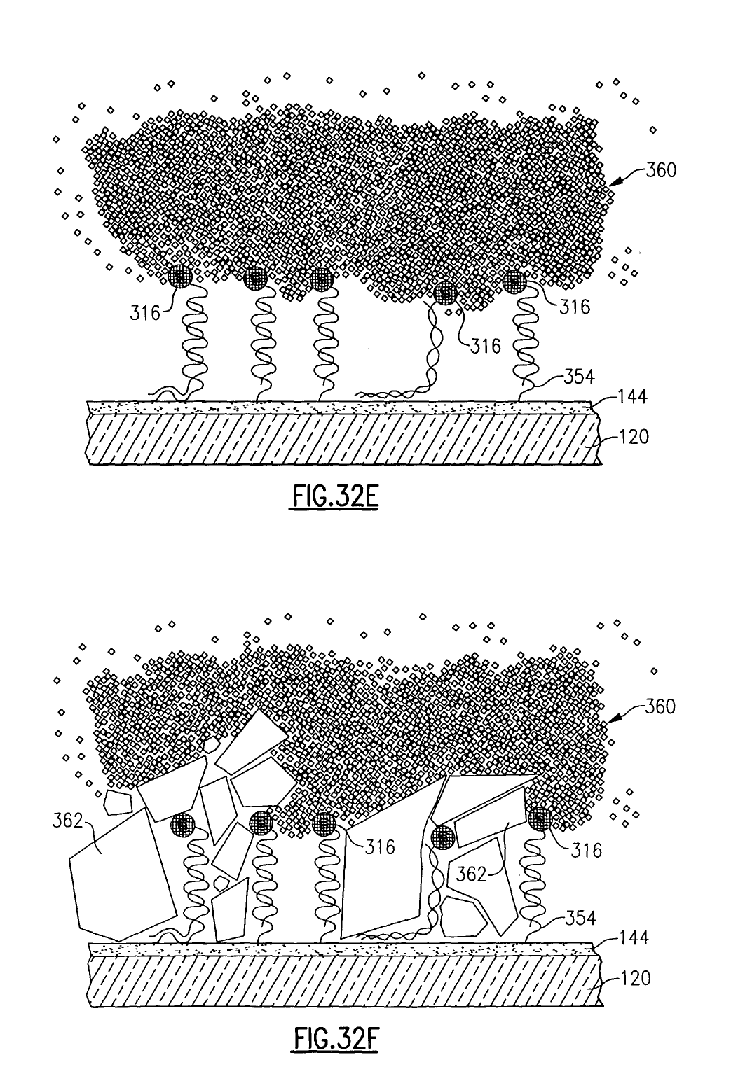

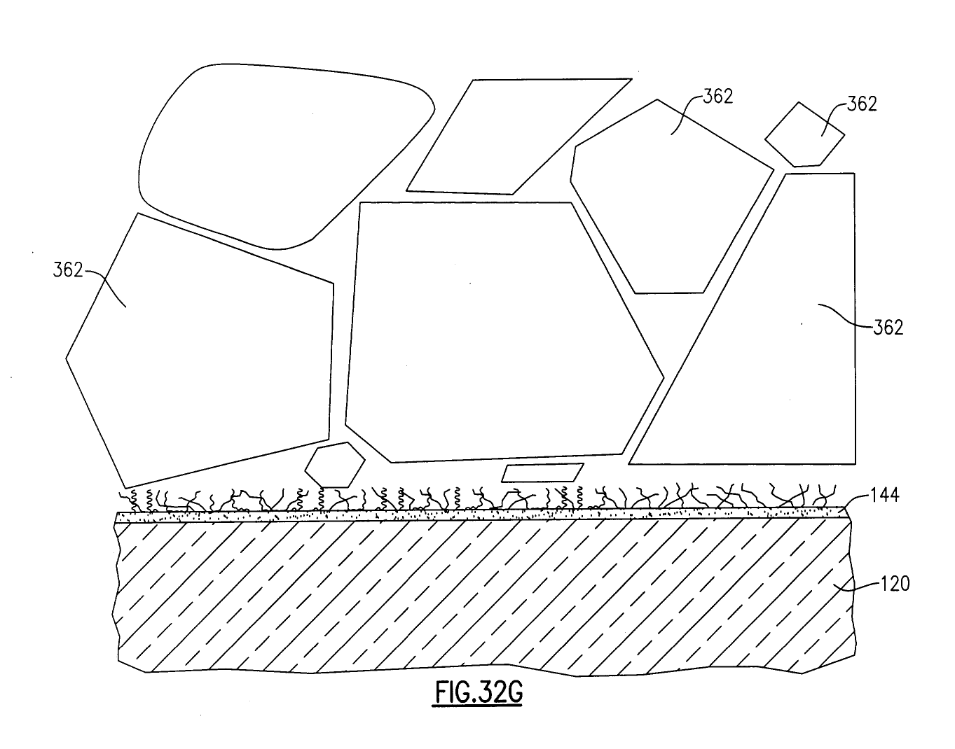

As would be apparent to those of skill in the art, in view of the present disclosure, the method for performing an immunochemical assay illustrated above in conjunction with FIGS. 31A-31G may also be implemented in genetic assays using DNA or RNA oligonucleotide sequences as the capture, target, and signal agents as discussed below in conjunction with FIGS. 32A-32G. In genetic assays, the bio-active nanoparticle is conjugated to an oligonucleotide signal probe. Further details relating to genetic assays on optical bio-disc are disclosed in co-pending and commonly assigned U.S. patent application Ser. No. 10/035,836 entitled “Surface Assembly for Immobilizing DNA Capture Probes and Bead-Based Assay Including Optical Bio-Discs and Methods Relating Thereto” filed Dec. 21, 2001; and U.S. patent application Ser. No. 10/150,702 entitled “Surface Assembly For Immobilizing DNA Capture Probes In Genetic Assays Using Enzymatic Reactions To Generate Signal In Optical Bio-Discs And Methods Relating Thereto” filed May 16, 2002. Both of which are herein incorporated by reference in their entireties.

Conjugation of the signal agent onto nanoparticles or nanocapsules may be achieved by passive absorption of the signal agent onto the nanoparticle or covalent binding of signal agents onto nanoparticles having free functional groups on its surface. The functional groups may include hydroxyl, carboxyl, aldehyde, sulfhydryl, maleimide, succinyl, anhydride, and amino functional groups. For example, monomers having carboxyl groups may be used to synthesize the nanoparticle resulting in a carboxy-modified nanoparticle having free carboxyl groups on its surface. The free carboxyl groups on the surface of the nanoparticle may be activated using N-hydroxysuccinimide (NHS) and 1-ethyl-3-(3-dimethylamino)propyl carbodiimide (CDI or EDAC) which generates an NHS ester active group. Once the NHS ester has been generated, free reactive amino groups on lysine residues of antibodies or the amino group on the PEG derivatized antibodies are then allowed to covalently bind to the carbon of the NHS ester in a substitution reaction. In this reaction, the nitrogen on the amino group on the antibodies acts as a nucleophile binding onto the carbon of the carboxyl group in the NHS ester removing the NHS leaving group thereby tethering the antibody on the surface of the nanoparticle. Further details relating to binding of biological material to functionalized surfaces are disclosed in commonly assigned and co-pending U.S. patent application Ser. No. 09/997,741 entitled “Dual Bead Assays Including Optical Biodiscs and Methods Relating Thereto” filed Nov. 27, 2001; U.S. patent application Ser. No. 10/038,297 entitled “Dual Bead Assays Including Covalent Linkages For Improved Specificity And Related Optical Analysis Discs” filed Jan. 4, 2002; U.S. patent application Ser. No. 10/086,941 entitled “Methods For DNA Conjugation Onto Solid Phase Including Related Optical Biodiscs and Disc Drive Systems” filed Feb. 26, 2002; and the above incorporated by reference U.S. patent application Ser. No. ______ entitled “Multi-Purpose Optical Analysis Disc for Conducting Assays and Related Methods for Attaching Capture Agents”. All of which are herein incorporated by reference in their entireties.

Use of the Bio-Active Nanoparticle in Optical Bio-Disc Genetic Assays

With reference now to FIG. 32A, there is illustrated a detailed partial cross sectional view of the target zone 140 showing the active layer 144 and the substrate 120 of the optical bio-disc 110 according to a DNA assay implementation of the present invention. In this embodiment, bio-active nanoparticle 316, having an enzyme 314 embedded therein, is linked to a signal DNA 356. In this method, the signal DNA 356 is non-complementary to a capture DNA 354, while a target RNA or DNA 358 contains separate sequences that are complementary to the signal DNA 356 and the capture DNA 354. In this embodiment, the target RNA or DNA 358 acts as a “bridge” to attach the signal DNA 356 to the capture DNA 354, as shown in FIG. 32B. This places the bio-active nanoparticle 316 in the target zone when the target RNA or DNA 358 is present. Altenatively, the target DNA or RNA 358 may be bound directly to the bio-active nanoparticle 316 such that when the target DNA or RNA 358 is hybridized with the capture DNA 354 on the target zone 140, the bio-active nanoparticle 316 is immobized within the target zone 140.

Referring to FIG. 32C, there is shown the addition of an enzyme substrate 348. Enzyme-substrate reaction 350 occurs as soon as the substrate comes in contact with the enzyme 314. The resulting enzyme-substrate reaction 350 produces a signal that is detectable by an optical disc reader. The signal generated may consist of precipitate formation, enzyme substrate luminescence, fluorescence, and/or enzyme substrate color change or formation.

Referring now to FIG. 32D, there is depicted the formation of an insoluble product 360 from the enzyme-substrate reaction 350. FIG. 32E further illustrates more massive amounts of insoluble product 360 formed by the enzyme reaction 350, which fill the capture or target zone 140.

Referring next to FIG. 32F, there is shown the insoluble product 360 aggregating and forming insoluble pellets or precipitates 362, which are deposited within the target zone 140. The active layer 144 may facilitate aggregation and deposition of the insoluble products 360, resulting in the formation of pellets comprising precipitates 362 that adhere to the active layer 144.

Next, FIG. 32G shows an expanded view of the complete aggregation of the insoluble product 360 forming large precipitate particles 362, relative to the DNA deposited on the target zone. These large aggregated particles or pellets 362 can then be detected using the optical disc reader 112 of the present invention.

The method for performing the genetic assay described above may be carried out on an open disc or inside a flow channel of the optical bio-disc. Further details regarding various methods for enzyme based detection of genetic materials on optical bio-discs are disclosed in the above referenced U.S. patent application Ser. Nos. 10/035,836 and 10/150,702. Furthermore, the genetic assays described above may be implemented using any of the bio-active hydrophobized nanoparticles, nanocapsules, and hydrophobized nanocapsules described above in conjunction with FIGS. 28A-28E when the appropriate enzyme is incorportated into the hydrophobized nanoparticle, nanocapsule, or hydrophobized nanocapsule of the present invention as would be apparent to one of skill in the art.

### TECHNICAL EXAMPLES

Having generally described the invention, the same will be more readily understood through reference to the following examples, which are provided by way of illustration, and are not intended to be limiting of the present invention.

Synthesis of Bio-Active Nanoparticles

### Example 1

### Synthesis of Bio-Active Nanoparticles Having Covalently Incorporated α-Chymotrypsin

Cross-linked polyacrylamide bio-active nanoparticles were prepared by polymerization of an aqueous solution of acryloylated α-chymotrypsin, acrylamide (Koch-Light, U.K.), and N—N′-methylenebisacrylamide (Reanal, Hungary), solubilized in a solution of AOT (Fluka, Germany) in purified toluene. Acryloylated α-chymotrypsin was produced by modification of α-chymotrypsin with acryloyl chloride (Serva, Germany) at 0° C. and pH 8.0. Toluene was purified by shaking with sulfuric acid and redistillation. Polymerization was initiated by UV-irradiation of degassed solution of reverse micelles containing solubilized acrylamide, methylene bisacrylamide, acryloylated α-chymotrypsin, and azobisisobutyronitrile (initiator for radical polymerization). UV-irradiation was perfomed for 10 minutes at a distance of 33 cm from the light source (XBO-200 xenon lamp, Germany). The nanoparticles were isolated from the organic solution by salting out with a 10-fold excess of cold acetone. The resulting bio-active nanoparticle may be dissolved in water or in an organic solvent with the addition of a micelle-forming surfactant and a small quantity of water as described above in conjunction with FIG. 29A.

As would be apparent to one of skill in the art, in view of the procedure described in this Example 1, any enzyme, antibody, DNA, receptors, ligands, or other labels including fluorescent dyes may be incorporated into the nanoparticle using the method described above.

Genetic Assays

### Example 2

### Spin Coating the Bio-Disc

Fresh polystyrene solution was prepared by adding 3 g polystyrene pellets (Sigma cat. no. 182427; molecular weight=280,000) to 310 ml toluene and stirring for 1 hour using a teflon stir bar and a stir plate. After the polystyrene was completely dissolved in the toluene, 68 ml reagent grade isopropanol was slowly added while stirring.

A stock solution of nitrocellulose was prepared by diluting a nitrocellulose collodium solution (4-8% in ethanol/diethylether, Fluka cat. no. 09986, lot no. 389973/1 30299) 1:5 in reagent grade ethanol. Prior to spin coating, the stock solution was diluted 1:10 with reagent grade ethanol and filtered using a 0.2 μm syringe filter.

A polycarbonate disc having a 200 Angstrom gold semi-reflective layer (BTI Optical Bio-disc Set FDL21:E001308) was placed on a “spin coater,” or modified centrifuge, with the reflective surface up. While rotating the disc on the spin coater, the reflective surface was cleaned with reagent grade alcohol.

The spin coater was set to start spinning at 2500 rpm, followed by acceleration to 4000 rpm within 10 seconds. During this 10-sec acceleration, a steady stream of polystyrene solution was applied to the disc using a pasteur pipette, with the polystyrene solution applied from the outer edge to the inner side in one smooth stroke.

The spin coater speed was then adjusted to 1500 rpm, and the diluted and filtered nitrocellulose solution was applied onto the inner portion of the disc in a steady stream using a pasteur pipette.

### Example 3

### Preparing the Bio-Disc for the Enzyme Assay

The disc from Example 2 was placed on a CD assembler/spindle with the nitrocellulose layer up. Between about 0.5 to 2.0 μl of 1 μM oligo probes (capture DNA) in 1 M NH₄OAc were applied to the disc at defined target zones. The droplets of capture DNA were dried onto the nitrocellulose at 37° C.

A cover disc containing U-shaped fluidic circuits (50 μM adhesive; Fraylock, DBL243a) was applied using a disc assembler spindle, and the disc was run through a wringer to seal the two discs.

### Example 4

### General Enzyme DNA Assay

DNA blocking solution (1% bovine serum albumin [BSA], 5× Denhardt's solution, 0.1 mg/ml salmon sperm DNA, 200 mM KCl, 10 mM MgCl₂, 50 mM Tris, pH 7.4) was degassed in a vacuum desiccator and injected into the fluidic circuits of a bio-disc prepared as in Example 3, taking care that no air bubbles remained in the circuits. The bio-disc was then incubated at room temperature for 30 to 60 minutes.

The DNA blocking solution was removed, and the fluidic circuits washed with hybridization buffer (200 mM NaCl, 10 mM MgCl₂, 50 mM Tris, pH 7.4) injected into the fluidic circuits using a syringe. PCR amplicons (target DNA amplified using biotinylated primer sets, purified using the Qiagen QIAquick PCR Purification Kit, cat. no. 28104, lot no. 10927932, and eluted using hybridization buffer) were denatured at 95° C. for 5 minutes and immediately placed on ice for 5 minutes.

The denatured amplicons were added to the appropriate fluidic circuits (10 μl per fluidic circuit) and allowed to hybridize for 1.5 to 2 hours at room temperature. Following hybridization, the fluidic circuits were washed with hybridization buffer using a syringe.

Neutravidin-Horseradish Peroxidase Conjugated enzyme (N-HRP; Pierce product no. 31001, lot no. BK46404) was diluted 1:5000 in hybridization buffer, and 12 μl was applied to each fluidic circuit. The disc was then incubated at room temperature for 15 minutes.

The fluidic circuits were then washed with hybridization buffer using a syringe, and 12 μl of TMB Substrate in Stable hydrogen peroxide buffer (Calbiochem cat. no. 613548, lot B34202) was added to each fluidic circuit. The enzyme reaction was allowed to proceed for 5 minutes, after which the reaction was stopped by flushing the fluidic circuits with distilled water using a syringe.

Each fluidic circuit was sealed with tape, and the bio-disc was then placed in a disc-reader, similar to that shown in FIG. 10, and scanned with a 780 nm lightbeam, with the light transmitted through the bio-disc at each target zone measured to detect changes in the amplitude of the transmitted light.

Alternatively, the Horse Radish Peroxidase enzyme (HRP) may be immobilized or embedded into the nanoparticles of the present invention as descibed above in Example 1. After the bio-active nanoparticle containing the HRP is synthesized, Neutravidin or Streptavidin is then bound or conjugated to the surface of the bio-active nanoparticle through passive adsorption or covalently if monomers having functional groups are used as described above. Once the Neutravidin is bound to the bio-active nanopaticles, the bio-active nanoparticles may then be used in the following examples of genetic assays.

### Example 5

### Enzyme DNA Assay Used to Identify Brucella Strains

A bio-disc with 6 target zones was prepared as in Example 3, with 1.6 μl of 10 μM DNA oligonucleotides specific to one of the Brucella strains applied to three of the target zones, as indicated in Table 2, below. One target zone contained a mix of all three Brucella species, one target zone contained biotinylated DNA (positive control), and one target zone contained no capture DNA (background).

Brucella sp. genomic DNA was subjected to PCR amplification using forward/reverse primer sets directed to B. melitensis to generate target B. melitensis amplicons. Each reaction contained 1 ng/μl Brucella DNA, 0.2 μM biotinylated forward and reverse primers, 0.2 mM dNTPs, 0.05 U/μl Taq polymerase, 3.0 mM MgCl₂ and 1×PCR buffer (Qiagen; 15 mM MgCl₂). The thermocycle conditions were:

| | |
|---|---|
| Step 1: | 95° C. for 12.5 minutes |
| Step 2: | 95° C. for 0.5 minute |
| Step 3: | 57° C. for 0.5 minute |
| Step 4: | 72° C. for 0.5 minute |
| Step 5: | Repeat Steps 2-4 34 times |
| Step 6: | 72° C. for 5.0 minutes |

The enzyme DNA assay was performed as described in Example 4, both without the addition of the target B. melitensis amplicons (results indicated in Table 2) and with the addition of purified target B. melitensis amplicons (results indicated in Table 3). Values from Tables 2 and 3 are event counts collected using an optical disc reader using the reflective disc format. The counting area was a rectangle, 940 μm×2300 μm; event count amplitude was between 225-500.

TABLE 2
No B. melitensis Amplicons

| | | | | | | Positive |
|---|---|---|---|---|---|---|
| | Bkgd | B. abortus | B. melitensis | B. suis | B. mix | Control |
| | 234 | 3338 | 723 | 1635 | 624 | 74696 |
| | 720 | 1906 | 343 | 401 | 630 | 106775 |
| Avg. | 477 | 2622 | 533 | 1018 | 627 | 90736 |
| SD | 344 | 1013 | 269 | 873 | 4 | 22683 |
| CV | 72% | 39% | 50% | 86% | 1% | 25% |

TABLE 3
Purified B. melitensis Amplicons

| | | | | | | Positive |
|---|---|---|---|---|---|---|
| | Bkgd | B. abortus | B. melitensis | B. suis | B. mix | Control |
| | 1179 | 5620 | 43185 | 2635 | 23020 | 65981 |
| | 693 | 5783 | 35862 | 2636 | 20792 | 68436 |
| | 754 | 4491 | 32250 | 2057 | 16263 | 81495 |
| | 394 | 5299 | 43165 | 2756 | 22929 | 46564 |
| Avg. | 755 | 5298 | 38616 | 2521 | 20751 | 65619 |
| SD | 323 | 575 | 5467 | 315 | 3164 | 14413 |
| CV | 43% | 11% | 14% | 12% | 15% | 22% |

As shown above, the B. melitensis target zone produces a much higher signal than the other Brucella species when the B. melitensis amplicon is used in the present invention.

### Example 6

### Concentration Dependent Detection of NosT Amplicon in Genetically Modified Plant Material

Bio-discs were prepared as in Example 3, with NosT 52-mer oligonucleotide capture probes applied to the target zones. NosT is a marker gene for genetically modified plant material.

GMO reference materials of soya bean powder containing different mass fractions of powder from genetically modified soya beans, were obtained from Fluka BioChemika, certified reference material IRMM-410R (0, 0.1, 0.5, 1, 2, 5% Roundup Ready Soya). DNA was extracted from each of the six reference materials, using the WIZARD® Magnetic DNA Purification System for Food (Promega, Madison, Wis.).

The extracted DNA was subjected to PCR amplification using biotinylated forward/reverse primer sets directed to NosT to generate biotinylated 280-mer target NosT amplicons. Each reaction contained 50 ng extracted DNA, 0.2 μM biotinylated forward and reverse primers, 0.2 mM dNTPs, 0.05 U/μl Taq polymerase, 3.0 mM MgCl₂ and 1×PCR buffer.

The enzyme assay was performed as in Example 4, using the NosT amplicons as target probes. The results are shown in Table 4.

TABLE 4
GMO Calibration Curve

| % GMO | Event Counts |
|---|---|
| 0 | 70 |
| 0.1 | 160 |
| 0.5 | 520 |
| 1 | 627 |
| 2 | 651 |
| 5 | 925 |

As shown in Table 4, the detectable signal (event counts) increased as the percentage of GMO material increased. Between 0% and 1% GMO, there was a 90% correlation between the amount of GMO material and the resulting signal.

Immunochemical Assays

The following immunochemical assays were perfomed using fluorescent microspheres as reporters. As discussed above, fluorescent nanoparticles may be synthesized by including fluorescent dyes in the polymerization mixture during synthesis of the nanoparticles. Hence, the examples described below in Examples 7, 8, 9, 10, and 11 may be perfomed using the fluorescent nanoparticles of the present invention.

### Example 7

### Direct Binding of Capture Antibodies on the Metal Layer

A 2 mg amount of affinity purified anti-HCG-alpha capture antibody (Biocheck, Burlingame, Calif.) was dissolved in 2% glycerol in PBS, pH 7.4 to obtain a 100 ug/ml stock solution. A pin stamper was used to directly apply multiple spots of 0.2-0.3 ul of the capture antibody stock solution on the gold metal layer (150 Angstroms thick) of the transmissive disc. The disc was then incubated in a humid environment using a humidity chamber at room temperature overnight. After incubation, the disc was washed with a gentle stream of deionized water to remove excess unbound capture antibodies and spun dried at 1000-1500 rpm. The cap portion having attached thereto the adhesive layer, having fluidic circuits formed therein, is then applied onto the substrate. After the disc is fully assembled, the fluidic channels are then filled with blocking buffer (10 ul/channel) containing 1% BSA, 1% Sucrose, 0.1% Tween-20 in PBS, pH 7.4. The disc is incubated for 2 hours to allow the blocking agents sufficient time to bind to unoccupied sites on the capture zone, cover disc, and substrate to prevent or minimize non-specific binding of reporter agents onto unwanted sites. Also, loosely bound capture antibodies will be displaced through dissociation of the antibody-antibody bonds by the detergent in the blocking buffer. After blocking, the excess blocking buffer is aspirated and the channels are filled with deionized water (10 ul/channel) to remove excess salt from the blocking buffer. The water is then aspirated and the disc is kept at 4 degrees Celsius prior to use.

### Example 8

### Purification of Microspheres

Microspheres may be purified using dialysis or centrifugation. With centrifugation, bead suspensions are centrifuged at a speed required to precipitate the particles. The speed is determined empirically and depends on the mass of the microspheres or nanoparticles and the density of the buffer containing the microspheres or nanoparticles [e.g., 0.2 um Fluospheres (Molecular Probes) in PBS or conjugation buffer may be centrifuged at 6000 rpm for 30 mins and 0.5 um Fluospheres (Molecular Probes) in PBS may be centrifuged at 14,000 rpm for 20 mins.). After the initial centrifugation of the bead suspension, the supernantant is discarded and the beads are resuspended in a conjugation buffer. The conjugation buffer is preferably a low ionic strength sodium phosphate buffer (PBS) having a pH slightly above the isoelectric point of the signal agent to be conjugated to the microspheres. The centrifugation, aspiration, and resuspension steps are repeated three times and the final pellet of beads is resuspended in conjugation buffer to obtain a suspension containing 10 mg/ml microspheres. The purified bead suspension is then stored at 4 degrees Celsius and sonicated for 30 seconds prior to use. Microspheres ranging in size from 0.01 um to 10 um in diameter and colloidal particles between 4 to 50 nm in diameter may be used in the present invention.

### Example 9

### Passive Adsorption of Signal Antibodies to 0.2 um Fluospheres

A 5.0 mg amount of purified and sonicated 0.2 um polystyrene carboxylate Fluospheres (Molecular Probes, Eugene, Oreg.), prepared as described in Example 8, were dispensed into 250 ul of 20 mM Sodium Phosphate buffer, pH 7.2 in a 1.7 ml Costar centrifuge tube. The beads were mixed in a vortex mixer and an additional 250 ul of Sodium Phosphate buffer was then added to the bead suspension. Then 250 ug of anti-HCG-beta was added to the bead suspension and immediately mixed using a vortex mixer. The tube containing the bead suspension was then placed on a Dynal mixer and rotated to 40 hours at 4 degrees Celsius shielded from light. After incubation, the beads were spun at 6000 rpm for 15 mins, the supernatant was aspirated and the pellet was resuspended with 500 ul of 20 mM Sodium Phosphate buffer, pH 7.2, sonicated for 30 seconds. After the initial washing step, the beads were further washed 3 times with 500 ul of 20 mM Sodium Phosphate buffer, pH 7.2 by repeated aspiration and spin cycles of 6000 rpm for 30 mins. The final pellet was then reconstituted with 1.0 ml 20 mM Sodium Phosphate buffer, pH 7.2 to obtain a final microsphere concentration of 5.0 mg/ml. The anti-HCG-beta conjugated microspheres were then stored at 4 degree Celsius.

### Example 10

### Conjugation of Anti-HCG-Beta to 0.5 um Fluospheres

A 400 ul bead suspension containing 4 mg of 0.5 um carboxylate polystyrene Fluospheres (Molecular Probes, Eugene, Oreg.) in PBS, prepared as described in Example 2, was dispensed into a 1.7 ml Costar centrifuge tube. Then 200 ug of anti-HCG-beta antibody in 15 mM potassium phosphate, 145 mM sodium chloride, pH 7.4 buffer was added to the bead suspension. The resulting antibody-bead suspension was then mixed using a Dynal rotator at room temperature for 4 hours. The suspension was then further incubated at 4 degrees Celsius without mixing for an additional 36 hours. After incubation, the beads were spun at 14000 rpm for 20 mins, the supernatant was aspirated and the pellet was resuspended with 500 ul of 20 mM Sodium Phosphate buffer, pH 7.2. After the initial washing step, the beads were further washed 3 times with 500 ul of 20 mM Sodium Phosphate buffer, pH 7.2. The final pellet was then reconstituted with 800 ul 20 mM Sodium Phosphate buffer, pH 7.2 containing 0.05% sodium azide. The anti-HCG-beta conjugated microspheres were then stored at 4 degree Celsius.

### Example 11

### HCG Assay Using the Optical Bio-Disc

The following materials were utilized in this Example: (1) Fully assembled optical bio-disc made according to Example 7; (2) HCG standard or unknown in 1% BSA PBS 7.4, 0.05% sodium azide; (3) Bead Conjugate Dilution Buffer (BCDB): 1% BSA, 0.1% Tween-20, and 0.05% sodium azide in PBS 7.4 (Note: The BSA concentration may be 0.1-10%; sucrose may be replaced with other sugars including glucose, fructose, trehalose, or lactose at a concentration of 0.1-10%; Tween-20 may be replaced with other non-ionic detergents including Triton X-100 and Tween-80 at a concentration of 0.1-5%; and sodium azide concentration may range from 0.01 to 1%); and (4) 0.2 um or 0.5 um Fluospheres conjugated with Anti-HCG-beta, respectively made according to either Example 9 or 10, washed and resuspended in BCDB.

The Assay: Various concentrations (0, 12.5, 25, 50, 250, and 500 mlU/ml) of HCG standard were mixed with an equal volume (10 ul) of 0.5 um Fluospheres conjugated with anti-HCG-beta in BCDB. Prior to use, the Fluospheres were washed and reconstituted in BCDB to obtain a bead concentration of 25 ug Fluospheres/ml of BCDB. The assay solutions were mixed and a 10 ul aliquot of each suspension was applied, using a pipette, through the inlet port into various channels in the bio-disc such as those shown and described above in conjunction with FIGS. 4 and 9. The disc containing the assay solutions was incubated at room temperature for 30 minutes. After incubation, the unbound beads and HCG were removed by spinning the disc at 2500 rpm for 6 minutes. This spin was enough to move all the liquid, containing unbound Fluospheres, out of the fluidic circuits to the inner and then to the outer peripheral circumferencial waste reservoir and into the absorber pads. After evacuating the fluidic circuits or channels, the amount of beads bound to the capture zones were quantified using a Molecular Dynamics Fluorescent Scanner model FluorImager 595. The results from this experiment are shown below in Table 5. The data presented below indicates that, for this particular experiment, the linear range of detection of HCG using the optical bio-disc is from 0 mlU/ml to 500 mlU/ml HCG when graphed in a semi-log format. The quantification of these beads may also be carried out using a fluorescent type optical disc reader or the optical disc reader as described above in conjunction with FIG. 10.

TABLE 5
Various Concentrations of HCG Standards Quantified Using
the Optical Bio-Disc of the Present Invention (Data are in
Relative Fluorescence Units)

| | HCG Concentration (mlU/ml) | | | | | |
|---|---|---|---|---|---|---|
| | 0 | 12.5 | 25 | 50 | 250 | 500 |
| Capture Zone 1 | 10468 | 11675 | 16002 | 16042 | 20610 | 23583 |
| 2 | 9869 | 11549 | 16388 | 17409 | 22868 | 25793 |
| 3 | 9869 | 12770 | 15079 | 18298 | 24475 | 26131 |
| Average | 10069 | 11998 | 15823 | 17250 | 22651 | 25169 |
| SD | 282 | 548 | 549 | 928 | 1585 | 1130 |
| % RSD | 2.8 | 4.6 | 3.5 | 5.4 | 7.0 | 4.5 |
| Background | 0 | 1930 | 5755 | 7181 | 12583 | 15101 |
| Subtracted | | | | | | |
| Data | | | | | | |

Synthesis of Nanoparticles

Enzyme immobilization on the nanoparticle can be achieved by immobilization during polymerization process (inside the particle) as described above in Example 1, or immobilization as a second step after polymerization is complete and nanoparticles are separated from the organic solvent (on the surface of the particle). Enzyme immobilization on the nanoparticle can be performed non-covalently or covalently. For covalent immobilization, active polymerizable groups of the enzyme molecule (such as double bonds and triple bonds), and active groups of monomers (double bonds and triple bonds) can be used.

An enzyme molecule can be chemically modified by attaching needed functional groups before solubilizing the enzyme into the RM solution as described below in Example 12.

### Example 12

### Modification of Enzyme With Double-Bond Containing Monomer

A 5 mL volume of Horse Radish Peroxidase (HRP) enzyme solution was prepared in 0.2 M phosphate buffer (pH=8.0). Then 10-20 uL of acryloylchloride was added to the enzyme solution at intensive stirring at 0° C., under a fumehood. The mixture was stirred for 20-30 min.

The degree of enzyme modification (amount of active monomers per enzyme molecule) was determined by titration of free amino groups of the enzyme, as described in Yakunitskaya, L. M. et al. Biokhimiya (“Biochemistry”), 1983, Vol. 48, No. 10, p.1596-1603 (in Russian).

### Example 13

### Preparation of Reverse Micellar Solutions Containing Monomers

A 0.5 M solution of AOT was prepared in toluene. Pre-calculated quantities of acrylamide, methacrylamide, and N,N′-methylene-bis-acrylamide, and 2-3 vol. % of water were added to the micellar solution. The resulting mixture was stirred until the monomers were fully dissolved. Then a polymerization initiator (azo-bis-isobutyronitrile) at 0.1 mg/mL concentration and a pre-determined amount of aqueous enzyme solution (non-modified enzyme or modified enzyme as in Example 12) was added into the mixture. The resulting solution was mixed until the solution became clear.

### Example 14

### Polymerization of Reverse Micellar Solutions Containing Monomers

The solution prepared in Example 13 was transfered into a quartz vial and degassed using a vacuum. The degassed solution was then irradiated with UV light for 5-40 min, at 33 cm distance between UV source and solution and 1 cm solution thickness. The degree of polymerization was monitored by NMR spectroscopy following the disappearance of “vinyl” protons peaks.

### Example 15

### Isolation of Synthesized Nanoparticles from Organic Solution

After the polymerization step in Example 14, 10-20 fold excess of cold (+4 C or below) acetone was added to the solution containing the newly synthesized nanoparticles or bio-active nanoparticles. The resulting mixture was centrifuged at 3000 rpm for 3-5 min. The supernatant was decanted. The precipitate was washed with cold acetone 2 times by repeated addition of cold acetone, centrifugation, and removal of the supernatant. The final precipitate was dried on air at room temperature for 10-30 min. The precipitate or nanoparticles was stored +4° C.

Concluding Summary

All patents, provisional applications, patent applications, technical specifications, and other publications mentioned in this specification are incorporated herein in their entireties by reference.

While this invention has been described in detail with reference to certain preferred embodiments, it should be appreciated that the present invention is not limited to those precise embodiments. Rather, in view of the present optical bio-system disclosure that describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

Furthermore, those skilled in the art will recognize, or be able to ascertain, using no more than routine experimentation, many equivalents to the specific embodiments of the invention described herein. Such equivalents are also intended to be encompassed by the following claims.

1. A method for making nanoparticles for use in optical bio-disc assays, said method comprising the steps of:
forming reverse micelles having an outer non-polar shell and an inner polar cavity by mixing a surfactant with a non-polar organic solvent;
solubilizing in said reverse micelles a polymerizing mixture comprising monomers and an initiator of polymerization; and
polymerizing said mixture.

2. The method according to claim 1 wherein said monomers are acrylic compounds that forms a linear polymer upon polymerization.

3. The method according to claim 1 wherein said monomers are methacrylic compounds that forms a cross-linked polymer when polymerized.

4. The method according to claim 1 wherein said surfactant is selected from the group comprising anionic, cationic, and non-ionic surfactants.

5. The method according to claim 1 wherein said surfactant is selected from the group comprising bis-(2-ethylhexyl) sulfosuccinate sodium salt, cetyltrimethylammonium bromide, polyethylene glycol dodecyl ether, polyethylene glycol oleyl ether, and any mixture thereof.

6. The method according to claim 1 wherein said non-polar organic solvent is selected from the group comprising octane, iso-octane, hexane, cyclohexane, toluene, chloroform, and mixtures thereof.

7. The method according to claim 1 wherein the inner polar cavity of the reverse micelle has diameter of about 1 to 200 nm.

8. The method according to claim 1 wherein said initiator of polymerization is azobisisobutyronitrile.

9. The method according to claim 1 wherein said polymerizing mixture includes labels selected from the group comprising fluorescent, luminescent, and infra-red dyes.

10. The method according to claim 9 wherein said labels are detectable using an optical disc drive.

11. The method according to claim 1 wherein said polymerizing mixture includes bio-active substances selected from the group comprising enzymes, antibodies, DNA, RNA, proteins, antigens, drugs, functionally active subunits, and parts and mixtures thereof.

12. The method according to claim 11 wherein said enzymes catalyze an enzyme-substrate reaction to produce a detectable signal.

13. The method according to claim 12 wherein said detectable signal is detectable using an optical disc drive.

14. The method according to claim 1 wherein said polymerizing mixture further includes a semiconductor material.

15. The method according to claim 1 wherein said polymerizing mixture further includes magnetic substances.

16. A method of testing for the presence of a target nucleic acid in a test sample, said method comprising the steps of:
providing a bio-disc having a substantially circular substrate with a center and an outer edge, an active layer associated with the substrate, a target zone disposed between the center and the outer edge, at least one strand of capture DNA having an affinity for the active layer such that the capture DNA is immobilized on the active layer in the target zone, the capture DNA and the target-nucleic acid having at least some complementary sequence;
depositing the test sample on the target zone;
allowing any target nucleic acid present in the test sample to hybridize with the capture-DNA;
providing a plurality of bio-active nanoparticles, said bio-active nanoparticles having an enzyme embedded therein;
binding the bio-active nanoparticles to the target nucleic acid such that bio-active nanoparticles bound to the target nucleic acid are immobilized within the target zone;
washing the target zone to remove any unbound bio-active nanoparticles;
depositing onto the target zone at least one enzyme substrate that reacts with the enzyme embeded in said bio-active nanoparticles to produce at least one detectable signal; and
detecting any signal in the target zone to thereby determine whether target-nucleic acid is present in the test sample.

17. A method of testing for the presence of a target nucleic acid in a test sample, said method comprising the steps of:
providing a bio-disc having a substantially circular substrate with a center and an outer edge, a target zone disposed between the center and the outer edge, at least one strand of capture DNA attached to the substrate in target zone, the capture DNA and the target nucleic acid having at least some complementary sequence;
depositing the test sample on the target zone;
allowing any target nucleic acid present in the test sample to hybridize with the capture-DNA;
providing a plurality of bio-active nanoparticles, said bio-active nanoparticles having an enzyme embedded therein and a signal DNA attached thereto;
hybridizing the signal DNA to the target nucleic acid such that the bio-active nanoparticles are immobilized within the target zone;
washing the target zone to remove any unbound bio-active nanoparticles;
depositing onto the target zone at least one enzyme substrate that reacts with the enzyme embeded in said bio-active nanoparticles to produce at least one detectable signal; and
detecting any signal in the target zone to thereby determine whether target-nucleic acid is present in the test sample.

18. A method for making hydrophobized nanoparticles for use in optical bio-disc assays, said method comprising the steps of:
forming reverse micelles having an outer non-polar shell and an inner polar cavity by contacting a mixture of micelle-forming surfactants and micelle-forming polymerizable surfactants with a non-polar organic solvent, said micelle-forming polymerizable surfactants being surfactants having a monomer attached thereto;
solubilizing in said reverse micelles a polymerizing mixture comprising monomers and an initiator of polymerization; and
polymerizing said monomers in said polymerizing mixture with said polymerizable surfactants thereby forming said hydrophobized nanoparticle.

19. The method according to claim 18 wherein said polymerizing mixture further includes bio-active substances selected from the group comprising enzymes, antibodies, DNA, RNA, proteins, antigens, drugs, functionally active subunits, and parts and mixtures thereof.

20. The method according to claim 19 wherein said enzymes catalyze an enzyme-substrate reaction to produce a detectable signal.

21. The method according to claim 20 wherein said detectable signal is detectable using an optical disc drive.

22. A method of using the nanoparticles made according to either claim 12 or 21 to test for the presence of a target nucleic acid in a test sample, said method of using comprising the steps of:
providing a bio-disc having a substantially circular substrate with a center and an outer edge, a target zone disposed between the center and the outer edge, at least one strand of capture DNA attached to the substrate in target zone, the capture DNA and the target nucleic acid having at least some complementary sequence;
depositing the test sample on the target zone;
allowing any target nucleic acid present in the test sample to hybridize with the capture-DNA;
attaching a signal DNA onto said nanoparticles;
hybridizing the signal DNA with the target nucleic acid bound to the capture DNA in the target zone thereby immobilizing the nanoparticles within the target zone;
washing the target zone to remove any unbound nanoparticles;
depositing onto the target zone at least one enzyme substrate that reacts with the enzyme embedded in said nanoparticles to produce at least one detectable signal; and
detecting any signal in the target zone to thereby determine whether target-nucleic acid is present in the test sample.

23. A method for making a nanocapsule for use in optical bio-disc assays, said method comprising the steps of:
forming reverse micelles having an outer non-polar shell and an inner polar cavity by mixing micelle-forming surfactants with a non-polar organic solvent;
adding weakly polar monomers that solubilized near the shell of the reverse micelles;
solubilizing in said reverse micelles an initiator of polymerization; and
polymerizing said weakly polar monomers to thereby form said nanocapsule.

24. A method for making a hydrophobized nanocapsule for use in optical bio-disc assays, said method comprising the steps of:
forming reverse micelles having an outer non-polar shell and an inner polar cavity by mixing micelle-forming polymerizable surfactants with a non-polar organic solvent, said micelle-forming polymerizable surfactants being surfactants having a monomer attached thereto;
solubilizing in said reverse micelles an initiator of polymerization; and
polymerizing said monomer such that the polymerizable surfactants are linked together forming the hydrophobized nanocapsule.

25. The method according to either claim 23 or 24 further comprising the step of solubilizing bio-active substances selected from the group comprising enzymes, antibodies, DNA, RNA, proteins, antigens, drugs, functionally active subunits, and parts and mixtures thereof, into said reverse micelles prior to polymerization.

26. The method according to claim 25 wherein said enzymes catalyze an enzyme-substrate reaction to produce a detectable signal.

27. The method according to claim 26 wherein said detectable signal is detectable using an optical disc drive.

28. A method of using the nanocapsule made according to claim 27 to test for the presence of a target nucleic acid in a test sample, said method of using comprising the steps of:
providing a bio-disc having a substantially circular substrate with a center and an outer edge, a target zone disposed between the center and the outer edge, at least one strand of capture DNA attached to the substrate in target zone, the capture DNA and the target nucleic acid having at least some complementary sequence;
depositing the test sample on the target zone;
allowing any target nucleic acid present in the test sample to hybridize with the capture-DNA;
attaching a signal DNA onto said nanocapsule;
depositing the nanocapsule on the target zone;
hybridizing the signal DNA to the target nucleic acid such that the nanocapsule is immobilized within the target zone;
washing the target zone to remove any unbound nanocapsule;
depositing onto the target zone at least one enzyme substrate that reacts with the enzyme inside said bio-active nanoparticle to produce at least one detectable signal; and
detecting any signal in the target zone to thereby determine whether target-nucleic acid is present in the test sample.

29. An optical assay disc implemented to perform any of the methods recited in any one of claims 1 to 21, 23, or 24.

30. Use of an optical analysis disc to perform any of the methods recited in any one of claims 1 to 21, 23, or 24.

31. An optical disc assembly made to perform any of the methods recited in any one of claims 1 to 21, 23, or 24.

32. An optical bio-disc system adapted to operate the optical assay disc recited in claim 29.

33. An optical bio-disc system adapted to read information stored on the optical assay disc recited in claim 29.

34. An optical bio-disc system adapted to write information relating to results of an assay onto the optical assay disc recited in claim 29.

35. An optical bio-disc system adapted to display on a monitor information relating to results of an assay conducted in association with the optical assay disc recited in claim 29.

36. An optical bio-disc system adapted to receive the optical assay disc recited in claim 29 and facilitate the performance of an assay associated with said optical assay disc.

37. An optical bio-disc system adapted to operate the optical analysis disc recited in claim 30.

38. An optical bio-disc system adapted to read information stored on the optical analysis disc recited in claim 30.

39. An optical bio-disc system adapted to write information relating to results of an assay onto the optical analysis disc recited in claim 30

40. An optical bio-disc system adapted to display on a monitor information relating to results of an assay conducted in association with the optical analysis disc recited in claim 30.

41. An optical bio-disc system adapted to receive the optical analysis disc recited in claim 30 and facilitate the performance of an assay associated with said optical analysis disc.

42. An optical bio-disc system adapted to operate the optical disc assembly recited in claim 31.

43. An optical bio-disc system adapted to read information stored on the optical disc assembly recited in claim 31.

44. An optical bio-disc system adapted to write information relating to results of an assay onto the optical disc assembly recited in claim 31.

45. An optical bio-disc system adapted to display on a monitor information relating to results of an assay conducted in association with the optical disc assembly recited in claim 31.

46. An optical bio-disc system adapted to receive the optical disc assembly recited in claim 31 and facilitate the performance of an assay associated with said optical disc assembly.
